(19) United States
(12) Patent Application Publication
(10) Pub. No.: US 2010/0316604 A1
(43) Pub. Date: Dec. 16, 2010

(54) INTERFERON LAMBDA FUSION POLYPEPTIDES

(75) Inventor: Peter ARTYMIUK, Sheffield (GB)

Correspondence Address: SPECKMAN LAW GROUP PLLC, 1201 THIRD AVENUE, SUITE 330, SEATTLE, WA, 98101, US

(73) Assignee: ASTERION LIMITED, Sheffield (GB)

(21) Appl. No.: 12/762,923

(22) Filed: Apr. 19, 2010

Related U.S. Application Data
(60) Provisional application No. 61/249,539, filed on Oct. 7, 2009.

(30) Foreign Application Priority Data
Apr. 21, 2009 (GB) GB0906810.7
Nov. 12, 2009 (GB) 0919753.4

(51) Int. Cl. A61K 38/21; C07K 14/555; C07H 21/00; A61P 35/00; A61P 31/14; A61P 31/20; A61P 31/12; C12N 15/63; C12N 5/10

(52) U.S. Cl. 424/85.4; 530/351; 536/23.4; 435/320.1; 435/325

(57) ABSTRACT

The disclosure relates to interferon lambda fusion polypeptides and dimers; nucleic acid molecules encoding said polypeptides and/or dimers, vectors and transformed cells including such nucleic acid molecules, pharmaceutical compositions including the interferon lambda fusion polypeptides and/or dimers, and methods of treating a disorder in a human subject by administering the polypeptides and/or dimers.

### REFERENCE TO RELATED APPLICATIONS

This application claims priority to U.S. provisional patent application no. 61/249,539, filed Oct. 7, 2009, and to United Kingdom patent applications no. GB0906810.7, filed Apr. 21, 2009, and GB0919753.4, filed Nov. 12, 2009.

### TECHNICAL FIELD OF THE INVENTION

The invention relates to interferon λ fusion polypeptides and dimers; nucleic acid molecules encoding said polypeptides and methods of treatment that use said polypeptides/dimers.

### BACKGROUND

The interferons represent a generic group of cytokines and are classified into three groups; type I, type II and type III. Each of the interferon groups has an associated anti-viral and anti-proliferative activity and therefore recombinant forms are used to treat both viral infection and cancer.

Type I interferon includes interferon α, interferon β, interferon ε, interferon κ and ω interferon. Interferon α (IFNA) is produced predominantly by B lymphocytes but also by macrophages and can be sub-divided into 13 sub-types. The level of homology between IFNA 1 isotypes is high being around 75-80% identity at the amino acid level. α interferon stimulates the activity of macrophages and Natural Killer (NK) cells to elicit an anti-viral response or an anti-tumour response.

Type II interferon includes one member, interferon γ and is involved in the regulation of immune and inflammatory responses. In humans interferon γ is encoded by a single gene and is produced by activated T-cells and NK cells. Interferon γ does have anti-viral and anti-tumour activity however this is generally weaker when compared to interferon α. The function of interferon γ is to enhance the effects of Type I interferon by recruiting leukocytes to a site of infection and by stimulating macrophages to engulf invading bacteria during an infection.

Type III interferon includes three interferon λ proteins referred to as IFN-λ1, IFN-λ2 and IFN-λ3 also known as IL29, IL28A and IL28B. Type III interferons have homology to type I interferons and IL10 and similar biological activity although they do not inhibit division of some B cell lines. Interferon λ1, λ2 and λ3 binds two dissimilar receptors referred to as interferon A receptor [INT-LR1] and interleukin 10 receptor 2 [IL10-R2].

The isolation and characterization of interferon A polypeptides is described in WO02/086087 and WO2005/023862.

It is known in the art to modify protein biopharmaceuticals to retard serum clearance. For example, Syed et al [Blood, 89, 3243-3252, (1997)] constructed an anti-coagulant fusion protein which fused hirudin with albumin. This fusion protein showed extended plasma half life whilst maintaining a potent anti-thrombin (anti-coagulant) activity. However a problem associated with this strategy is that hirudin is a foreign protein which is known to provoke a strong immune response. Similarly, blood clotting Factor VII and VIIa has been fused to albumin to extend serum half life [see WO2007/090584 and Weimer et al Thromb Haemost, 99: 659-667, (2008)].

A further method to increase the effective molecular weight of proteins and to produce a product which has reduced immunogenicity is to coat the protein in polyethylene glycol (PEG). The in-vivo half-life of GH has been increased by conjugating the GH with polyethylene glycol, which is termed “pegylation” [see Abuchowski et al., J. Biol Chem., 252:3582-3586 (1977)]. PEG is typically characterised as a non-immunogenic uncharged polymer. PEG is believed to slow renal clearance by providing increased hydrodynamic volume in pegylated proteins (Maxfield et al., Polymer, 16:505-509 (1975)). U.S. Pat. No. 5,849,535 also describe human GH (hGH) variants which are conjugated to one or more polyols. A problem associated with pegylation of GH is that the affinity of pegylated GH for its receptor is reduced thereby requiring adminstration of high dosage regimes. This also applies to other pegylated proteins, for example granulocyte colony stimulating hormone [GCSF], the interferons, see WO2005/023862 and somatostatin.

### SUMMARY

This disclosure relates to interferon λ recombinant forms and including glycosylated forms that have improved pharmacokinetics and activity.

According to an aspect of the invention there is provided a fusion polypeptide comprising:

the amino acid sequence of an interferon λ, or an amino acid sequence variant thereof, linked, directly or indirectly, to the binding domain of an interferon λ receptor, or an amino acid sequence variant thereof.

An interferon λ interferon receptor amino sequence variant may differ in amino acid sequence by one or more substitutions, additions, deletions, truncations which may be present in any combination. Among preferred variants are those that vary from a reference polypeptide by substitution of at least one cysteine amino acid residue from either interferon λ and/or the interferon λ receptor. Most highly preferred are variants which retain the same biological function and activity as the reference polypeptide from which it varies. Preferably the variant has improved biological activity.

In a preferred embodiment of the invention, the variant polypeptide varies by substitution of at least one cysteine amino acid residue wherein said cysteine amino acid is substituted for an amino acid selected from the group consisting of: aspartic acid, glutamic acid, phenylalanine, glycine, histidine, isoleucine, lysine, leucine, methionine, proline, glutamine, arginine, threonine, valine, tryptophan or tyrosine. Preferably said cysteine substitution is for serine, alanine or asparagine.

In a preferred embodiment of the invention said interferon λ is linked to the binding domain of the of the interferon λ receptor by a peptide linker. Preferably a flexible peptide linker. Preferably said peptide linking molecule comprises at least 1, 2, 3, 4, 5 or 6 copies of the peptide Gly Gly Gly Gly Ser (SEQ ID NO: 188).

In a preferred embodiment of the invention said peptide linking molecule consists of 4 or 5 copies of the peptide Gly Gly Gly Gly Ser (SEQ ID NO: 188).

In an alternative preferred embodiment of the invention said peptide linker molecule comprises or consists of one copy of the glycosylation motif Asn-Xaa-Ser or Asn-Xaa-Thr where X is any amino acid except proline.

In a preferred embodiment of the invention said peptide linker molecule comprises at least 5 amino acid residues.

In a preferred embodiment of the invention said peptide linker comprises 5-50 amino acid residues.

In a further preferred embodiment of the invention said peptide linker consists of 5, 10, 15, 20, 25, 30, 35, 40, 45 or 50 amino acid residues.

In a preferred embodiment of the invention said peptide linker molecule comprises at least one copy of the motif (Xaa₁ Xaa₂ Xaa₃ Xaa₄ Xaa₅; SEQ ID NO: 189) wherein said motif comprises the glycosylation motif Asn-Xaa-Ser or Asn-Xaa-Thr.

In a preferred embodiment of the invention said peptide linker comprises at least one copy of an amino acid motif selected from the group consisting of:

Asn₁-Xaa₂-Ser₃ Xaa₄ Xaa₅ (SEQ ID NO: 190) wherein Xaa₂ is any amino acid except proline;
Xaa₁ Asn₂-Xaa₃-Ser₄ Xaa₅ (SEQ ID NO: 191) wherein Xaa₃ is any amino acid except proline;
Xaa₁ Xaa₂ Asn₃-Xaa₄-Ser₅ (SEQ ID NO: 192) wherein Xaa₄ is any amino acid except proline;
Asn₁-Xaa₂-Thr₃ Xaa₄ Xaa₅ (SEQ ID NO: 193) wherein Xaa₂ is any amino acid except proline;
Xaa₁ Asn₂-Xaa₃-Thr₄ Xaa₅ (SEQ ID NO: 194) wherein Xaa₃ is any amino acid except proline; and
Xaa₁ Xaa₂ Asn₃-Xaa₄-Thr₅ (SEQ ID NO: 195) wherein Xaa₄ is any amino acid except proline.

Preferably said peptide linker comprises at least one copy of a motif selected from the group consisting of:

Asn₁-Xaa₂-Ser₃ Gly₄ Ser₅ (SEQ ID NO: 196) wherein Xaa₂ is any amino acid except proline;
Gly₁ Asn₂-Xaa₃-Ser₄ Ser₅ (SEQ ID NO: 197) wherein Xaa₃ is any amino acid except proline;
Gly₁ Gly₂ Asn₃-Xaa₄-Ser₅ (SEQ ID NO: 198) wherein Xaa₄ is any amino acid except proline;
Asn₁-Xaa₂-Thr₃ Gly₄ Ser₅ (SEQ ID NO: 199) wherein Xaa₂ is any amino acid except proline;
Gly₁ Asn₂-Xaa₃-Thr₄ Ser₅ (SEQ ID NO: 200) wherein Xaa₃ is any amino acid except proline; and
Gly₁ Gly₂ Asn₃-Xaa₄-Thr₅ (SEQ ID NO: 201) wherein Xaa₄ is any amino acid except proline.

In an alternative preferred embodiment of the invention said peptide linker comprises at least one copy of a motif selected from the group consisting of:

Asn₁-Xaa₂-Ser₃ Ser₄ Gly₅ (SEQ ID NO: 202) wherein Xaa₂ is any amino acid except proline;
Ser₁ Asn₂-Xaa₃-Ser₄ Gly₅ (SEQ ID NO: 203) wherein Xaa₃ is any amino acid except proline;
Ser₁ Ser₂ Asn₃-Xaa₄-Ser₅ (SEQ ID NO: 204) wherein Xaa₄ is any amino acid except proline;
Asn₁-Xaa₂-Thr₃ Ser₄ Gly₅ (SEQ ID NO: 205) wherein Xaa₂ is any amino acid except proline;
Ser₁ Asn₂-Xaa₃-Thr₄ Gly₅ (SEQ ID NO: 206) wherein Xaa₃ is any amino acid except proline; and
Ser₁ Ser₂ Asn₃-Xaa₄-Thr₅ (SEQ ID NO: 207) wherein Xaa₄ is any amino acid except proline.

In a preferred embodiment of the invention said peptide linker molecule comprises at least one copy of the motif (Xaa₁ Xaa₂ Xaa₃ Xaa₄ Xaa₅; SEQ ID NO: 189) wherein said motif comprises the glycosylation motif Asn-Xaa-Ser or Asn-Xaa-Thr and at least one copy of the motif (Gly Gly Gly Gly Ser; SEQ ID NO: 188) wherein said peptide linker is 5-50 amino acids.

In a preferred embodiment of the invention said peptide linker comprises at least one copy of the motif (Xaa₁ Xaa₂ Xaa₃ Xaa₄ Xaa₅; SEQ ID NO: 189) wherein said motif comprises the glycosylation motif Asn-Xaa-Ser or Asn-Xaa-Thr and a copy of the motif (Ser Ser Ser Ser Gly; SEQ ID NO: 188) wherein said peptide linker is 5-50 amino acids.

In a preferred embodiment of the invention said fusion polypeptide linker is modified by the addition of at least one sugar selected from the group consisting of: mannose, galactose, n-acetyl glucosamine, n-acetyl neuraminic, acid n-glycolyl neuraminic acid, n-acetyl galactosamine, fucose, glucose, rhamnose, xylose, or a combinations of sugars, for example in an oligosacharide or scaffolded system.

Suitable carbohydrate moieties include monosaccharides, oligosaccharides and polysaccharides, and include any carbohydrate moiety that is present in naturally occurring glycoproteins or in biological systems. For example, optionally protected glycosyl or glycoside derivatives, for example optionally-protected glucosyl, glucoside, galactosyl or galactoside derivatives. Glycosyl and glycoside groups include both α and β groups. Suitable carbohydrate moieties include glucose, galactose, fucose, GlcNAc, GalNAc, sialic acid, and mannose, and oligosaccharides or polysaccharides comprising at least one glucose, galactose, fucose, GlcNAc, GalNAc, sialic acid, and/or mannose residue.

Any functional groups in the carbohydrate moiety may optionally be protected using protecting groups known in the art (see for example Greene et al, “Protecting groups in organic synthesis”, 2nd Edition, Wiley, New York, 1991, the disclosure of which is hereby incorporated by reference). Suitable protecting groups for any —OH groups in the carbohydrate moiety include acetate (Ac), benzyl (Bn), silyl (for example tert-butyl dimethylsilyl (TBDMSi) and tert-butyldiphenylsilyl (TMDPSi)), acetals, ketals, and methoxymethyl (MOM). Any protecting groups may be removed before or after attachment of the carbohydrate moiety to the peptide linker.

In a preferred embodiment of the invention said sugars are unprotected.

Particularly preferred carbohydrate moieties include Glc(Ac)₄β-, Glc(Bn)₄β-, Gal(Ac)₄β-, Gal(Bn)₄β-, Glc(Ac)₄α(1,4)Glc(Ac)₃α(1,4)Glc(Ac)₄β-, β-Glc, β-Gal, -Et-β-Gal,-Et-β-Glc, Et-α-Glc, -Et-α-Man, -Et-Lac, -β-Glc(Ac)₂, β-Glc(Ac)₃, -Et-α-Glc(Ac)₂, -Et-α-Glc(Ac)₃, -Et-α-Glc(Ac)₄, -Et-β-Glc(Ac)₂, -Et-β-Glc(Ac)₃, -Et-β-Glc(Ac)₄, -Et-α-Man(Ac)₃, -Et-α-Man(Ac)₄, -Et-β-Gal(Ac)₃, -Et-13-Gal(Ac)₄, -Et-Lac(Ac)₅, -Et-Lac(Ac)₆, -Et-Lac(Ac)₇, and their deprotected equivalents.

Preferably, any saccharide units making up the carbohydrate moiety which are derived from naturally occurring sugars will each be in the naturally occurring enantiomeric form, which may be either the D-form (e.g. D-glucose or D-galactose), or the L-form (e.g. L-rhamnose or L-fucose). Any anomeric linkages may be α- or β- linkages.

In an alternative preferred embodiment of the invention said polypeptide does not comprise a peptide linker molecule and is a direct fusion of interferon λ and the interferon λ binding domain of the interferon λ receptor.

In a preferred embodiment of the invention said fusion polypeptide comprises the amino acid sequence as represented in FIG. 1 (SEQ ID NO: 1), FIG. 2 (SEQ ID NO: 2) or FIG. 3 (SEQ ID NO: 3) wherein said polypeptide optionally includes a signal sequence.

In a preferred embodiment of the invention interferon λ is represented by FIG. 1 (SEQ ID NO: 1).

In a preferred embodiment of the invention said interferon λ amino acid sequence variant is a modification of amino acid residue cysteine 190 in FIG. 1 (SEQ ID NO: 1); preferably cysteine190 is modified by an amino acid substitution.

In a preferred embodiment of the invention said substitution is cysteine 190 for a serine amino acid.

In an alternative preferred embodiment of the invention said amino acid sequence variant is a modification of amino acid residue cysteine 34 in FIG. 1 (SEQ ID NO: 1); preferably cysteine 34 is modified by an amino acid substitution.

In a preferred embodiment of the invention said substitution is cysteine 34 for a serine amino acid.

In a preferred embodiment of the invention said interferon λ amino acid sequence variant is a modification of amino acid residue 190 and amino acid residue 34.

In a preferred embodiment of the invention interferon λ is represented by FIG. 2 (SEQ ID NO: 2).

In a preferred embodiment of the invention interferon λ is represented by FIG. 3 (SEQ ID NO: 3).

In a preferred embodiment of the invention said interferon λ amino acid sequence variant is a modification of amino acid residue cysteine 73 in FIG. 2 or 3 (SEQ ID NO: 2 or 3); preferably cysteine 73 is modified by an amino acid substitution.

In a preferred embodiment of the invention said substitution is cysteine 73 for a serine amino acid.

In a preferred embodiment of the invention said substitution is cysteine 73 for an alanine amino acid.

In a preferred embodiment of the invention said interferon λ amino acid sequence variant is a modification of amino acid residue cysteine 75 in FIG. 2 or 3 (SEQ ID NO: 2 or 3); preferably cysteine 75 is modified by an amino acid substitution.

In a preferred embodiment of the invention said substitution is cysteine 75 for a serine amino acid.

In a preferred embodiment of the invention the interferon binding domain of an interferon receptor is an interferon λ receptor binding domain comprising FIG. 4a, FIG. 4b or FIG. 4c (SEQ ID NO: 4, 5 or 6) wherein said polypeptide optionally includes a signal sequence.

In a preferred embodiment of the invention interferon λ is linked to an interferon λ binding domain of an interferon λ receptor wherein said interferon λ is positioned amino terminal to said binding domain in said fusion polypeptide.

In an alternative preferred embodiment of the invention interferon λ is linked to an interferon λ binding domain of an interferon λ receptor wherein said interferon is positioned carboxyl-terminal to said binding domain in said fusion polypeptide.

In a preferred embodiment of the invention said fusion polypeptide comprises an amino acid sequence as represented in FIG. 6a, 6b, 6c, 6d, 6e, 6f, 6h, 6i, 6j, 6k or 6l (SEQ ID NO: 10-21), wherein said polypeptide optionally includes a signal sequence.

In a preferred embodiment of the invention said fusion polypeptide comprises an amino acid sequence as represented in FIG. 7a, 7b, 7c, 7d, 7e, 7f, 7h, 7i, 7j, 7k or 7l (SEQ ID NO: 22-33), wherein said polypeptide optionally includes a signal sequence.

In a preferred embodiment of the invention said fusion polypeptide comprises an amino acid sequence as represented in FIG. 8a, 8b, 8c, 8d, 8e, 8f, 8h, 8i, 8j, 8k or 8l (SEQ ID NO: 34-45), wherein said polypeptide optionally includes a signal sequence.

In a preferred embodiment of the invention said fusion polypeptide comprises an amino acid sequence as represented in FIG. 9a, 9b, 9c, 9d, 9e, 9f, 9h, 9i, 9j, 9k or 9l (SEQ ID NO: 46-57), wherein said polypeptide optionally includes a signal sequence.

In a preferred embodiment of the invention said fusion polypeptide comprises an amino acid sequence as represented in FIG. 10a, 10b, 10c, 10d, 10e, 10f, 10h, 10i, 10j, 10k, 10l, 10m, 10n, 10o or 10p (SEQ ID NO: 58-73), wherein said polypeptide optionally includes a signal sequence.

In a preferred embodiment of the invention said fusion polypeptide comprises an amino acid sequence as represented in FIG. 11a, 11b, 11c, 11d, 11e, 11f, 11h, 11i, 11j, 11k, 11l, 11m, 11n, 11o or 11p (SEQ ID NO: 74-89), wherein said polypeptide optionally includes a signal sequence.

In a preferred embodiment of the invention said fusion polypeptide comprises an amino acid sequence as represented in FIG. 12a, 12b, 12c, 12d, 12e, 12f, 12h, 12i, 12j, 12k, 12l, 12m, 12n, 12o or 12p (SEQ ID NO: 90-105), wherein said polypeptide optionally includes a signal sequence.

In a preferred embodiment of the invention said fusion polypeptide comprises an amino acid sequence as represented in FIG. 13a, 13b, 13c, 13d, 13e, 13f, 13h, 13i, 13j, 13k, 13l, 13m, 13n, 13o or 13p (SEQ ID NO: 106-121), wherein said polypeptide optionally includes a signal sequence.

In a preferred embodiment of the invention said fusion polypeptide comprises an amino acid sequence as represented in FIG. 14a, 14b, 14c, 14d, 14e, 14f, 14h, 14i, 14j, 14k, 14l, 14m, 14n, 14o or 14p (SEQ ID NO: 122-137), wherein said polypeptide optionally includes a signal sequence.

In a preferred embodiment of the invention said fusion polypeptide comprises an amino acid sequence as represented in FIG. 15a, 15b, 15c, 15d, 15e, 15f, 15h, 15i, 15j, 15k, 15l, 15m, 15n, 15o or 15p (SEQ ID NO: 138-153), wherein said polypeptide optionally includes a signal sequence.

In a preferred embodiment of the invention said fusion polypeptide comprises an amino acid sequence as represented in FIG. 16a, 16b, 16c, 16d, 16e, 16f, 16h, 16i, 16j, 16k, 16l, 16m, 16n, 16o or 16p (SEQ ID NO: 154-169), wherein said polypeptide optionally includes a signal sequence.

In a preferred embodiment of the invention said fusion polypeptide comprises an amino acid sequence as represented in FIG. 17a, 17b, 17c, 17d, 17e, 17f, 17h, 17i, 17j, 17k, 17l, 17m, 17n, 17o or 17p (SEQ ID NO: 170-181), wherein said polypeptide optionally includes a signal sequence.

According to an aspect of the invention there is provided a homodimer consisting of two polypeptides wherein each of said polypeptides comprises:
i) a first part comprising interferon λ, or a receptor binding domain thereof, optionally linked by a peptide linking molecule to
ii) a second part comprising at least one interferon λ binding domain or part thereof, of an interferon λ receptor.

According to an aspect of the invention there is provided a nucleic acid molecule that encodes a polypeptide according to the invention.

According to a further aspect of the invention there is provided a vector comprising a nucleic acid molecule according to the invention.

In a preferred embodiment of the invention said vector is an expression vector adapted to express the nucleic acid molecule according to the invention.

A vector including nucleic acid (s) according to the invention need not include a promoter or other regulatory sequence, particularly if the vector is to be used to introduce the nucleic acid into cells for recombination into the genome for stable transfection. Preferably the nucleic acid in the vector is operably linked to an appropriate promoter or other regulatory elements for transcription in a host cell. The vector may be a bi-functional expression vector which functions in multiple hosts. By “promoter” is meant a nucleotide sequence upstream from the transcriptional initiation site and which contains all the regulatory regions required for transcription. Suitable promoters include constitutive, tissue-specific, inducible, developmental or other promoters for expression in eukaryotic or prokaryotic cells. “Operably linked” means joined as part of the same nucleic acid molecule, suitably positioned and oriented for transcription to be initiated from the promoter. DNA operably linked to a promoter is “under transcriptional initiation regulation” of the promoter.

In a preferred embodiment the promoter is a constitutive, an inducible or regulatable promoter.

According to a further aspect of the invention there is provided a cell transfected or transformed with a nucleic acid molecule or vector according to the invention.

Preferably said cell is a eukaryotic cell. Alternatively said cell is a prokaryotic cell.

In a preferred embodiment of the invention said cell is selected from the group consisting of; a fungal cell (e.g. Pichia spp, Saccharomyces spp, Neurospora spp); insect cell (e.g. Spodoptera spp); a mammalian cell (e.g. COS cell, CHO cell); a plant cell.

According to a further aspect of the invention there is provided a pharmaceutical composition comprising a polypeptide according to the invention including an excipient or carrier.

In a preferred embodiment of the invention said pharmaceutical composition is combined with a further therapeutic agent.

When administered the pharmaceutical composition of the present invention is administered in pharmaceutically acceptable preparations. Such preparations may routinely contain pharmaceutically acceptable concentrations of salt, buffering agents, preservatives, compatible carriers, and optionally other therapeutic agents.

The pharmaceutical compositions of the invention can be administered by any conventional route, including injection. The administration and application may, for example, be oral, intravenous, intraperitoneal, intramuscular, intracavity, intra-articuar, subcutaneous, topical (eyes), dermal (e.g a cream lipid soluble insert into skin or mucus membrane), transdermal, or intranasal.

Pharmaceutical compositions of the invention are administered in effective amounts. An “effective amount” is that amount of pharmaceuticals/compositions that alone, or together with further doses or synergistic drugs, produces the desired response. This may involve only slowing the progression of the disease temporarily, although more preferably, it involves halting the progression of the disease permanently. This can be monitored by routine methods or can be monitored according to diagnostic methods.

The doses of the pharmaceuticals compositions administered to a subject can be chosen in accordance with different parameters, in particular in accordance with the mode of administration used and the state of the subject (i.e. age, sex). When administered, the pharmaceutical compositions of the invention are applied in pharmaceutically-acceptable amounts and in pharmaceutically-acceptable compositions. When used in medicine salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The pharmaceutical compositions may be combined, if desired, with a pharmaceutically-acceptable carrier. The term “pharmaceutically-acceptable carrier” as used herein means one or more compatible solid or liquid fillers, diluents or encapsulating substances that are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction that would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier that constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as syrup, elixir or an emulsion.

Compositions suitable for parenteral administration conveniently comprise a sterile aqueous or non-aqueous preparation that is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

According to a further aspect of the invention there is provided a method to treat a human subject suffering from a viral infection comprising administering an effective amount of a polypeptide according to the invention.

According to a further aspect of the invention there is provided a method to treat a human subject suffering from cancer comprising administering an effective amount of a polypeptide according to the invention.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting, for example, lung, breast, thyroid, lymphoid, gastrointestinal, and genito-urinary tract, as well as adenocarcinomas which include malignancies such as most colon cancers, renal-cell carcinoma, prostate cancer and/or testicular tumours, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumours composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumours of mesenchymal derivation.

In a further preferred embodiment of the invention said polypeptide is administered at two day intervals; preferably said polypeptide is administered at weekly, 2 weekly or monthly intervals.

According to an aspect of the invention there is provided the use of a polypeptide according to the invention for the manufacture of a medicament for the treatment of viral infection.

According to an aspect of the invention there is provided the use of a polypeptide according to the invention for the manufacture of a medicament for the treatment of cancer.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### BRIEF DESCRIPTION OF THE FIGURES

An embodiment of the invention will now be described by example only and with reference to the following figures:

FIG. 1(a) is the amino acid sequence of interferon λ 1 [IL29] (SEQ ID NO: 1); FIG. 1(b) is the amino acid sequence of interferon λ 1 [IL29] highlighting cysteine amino acid residues;

FIG. 2(a) is the amino acid sequence of interferon λ 2 [IL28A] (SEQ ID NO: 2); FIG. 2(b) is the amino acid sequence of interferon λ 2 [IL28A] highlighting cysteine amino acid residues;

FIG. 3(a) is the amino acid sequence of interferon λ 3 [IL28B] (SEQ ID NO: 3); FIG. 3(b) is the amino acid sequence of interferon λ 3 [IL28B] highlighting cysteine amino acid residues;

FIG. 4(a) Interferon lambda receptor 1 IFNLR1 (high affinity receptor) (SEQ ID NO: 4); FIG. 4(b) Interferon lambda receptor 1 IFNLR1 (high affinity receptor chain) ectodomain (SEQ ID NO: 5); FIG. 4(c) Interferon lambda receptor 1 IFNLR1 (high affinity receptor chain) ectodomain with mutations C86X&C150X (SEQ ID NO: 6);

FIG. 5(a) 1IL10R2 receptor (low affinity receptor chain) (SEQ ID NO: 7); FIG. 5(b) IL10R2 receptor (low affinity receptor chain) ectodomain (SEQ ID NO: 8); FIG. 5(c) IL10R2 receptor (low affinity receptor chain) ectodomain with C106X mutation (SEQ ID NO: 9);

FIGS. 6(a) to 6(l) are the amino acid sequences of IL29-(G4S)5-IFNLR1ect LR fusions (SEQ ID NO: 10-21);

FIGS. 7(a) to 7(l) are the amino acid sequences of IL29-(G4S)4-IL10R2ect LR fusions (SEQ ID NO: 22-33);

FIGS. 8(a) to 8(l) are the amino acid sequences of IFNLR1ect-(G4S)4-IL29 RL fusions (SEQ ID NO: 34-45);

FIGS. 9(a) to 9(l) are the amino acid sequences of IL10R2ect-(G4S)5-IL29 RL fusions (SEQ ID NO: 46-57);

FIGS. 10(a) to 10(p) are the amino acid sequences of IL28A-(G4S)5-IFNLR1ect LR fusions (SEQ ID NO: 58-73);

FIGS. 11(a) to 11(p) are the amino acid sequences of IL28A-(G4S)4-IL10R2ect LR fusions (SEQ ID NO: 74-89);

FIGS. 12(a) to 12(p) are the amino acid sequences of IFNLR1ect-(G4S)4-IL28A RL fusions (SEQ ID NO: 90-105);

FIGS. 13(a) to 13(p) are the amino acid sequences of IL10R2ect-(G4S)5-IL28A RL fusions (SEQ ID NO: 106-121);

FIGS. 14(a) to 14(p) are the amino acid sequences of IL28b-(G4S)5-IFNLR1ect LR fusions (SEQ ID NO: 122-137);

FIGS. 15(a) to 15(p) are the amino acid sequences of IL28b-(G4S)4-IL10R2ect LR fusions (SEQ ID NO: 138-153);

FIGS. 16(a) to 16(p) are the amino acid sequences of IFNLR1ect-(G4S)4-IL28b RL fusions (SEQ ID NO: 154-169);

FIGS. 17(a) to 17(p) are the amino acid sequences of IL10R2ect-(G4S)5-IL28b RL fusions (SEQ ID NO: 170-185);

FIG. 18a is the amino acid sequence of IL28B (C73A)-(G4S)5-IFNLR1 LR fusion (SEQ ID NO: 186); FIG. 18b is the amino acid sequence of IL28B (C73A)-(G4S)4-IL10R2ect LR fusion (SEQ ID NO: 187); and

FIGS. 19A and B show the transient and stable expression of fusion proteins 13E1 and 13F1 (SEQ ID NO: 186 and 187), respectively.

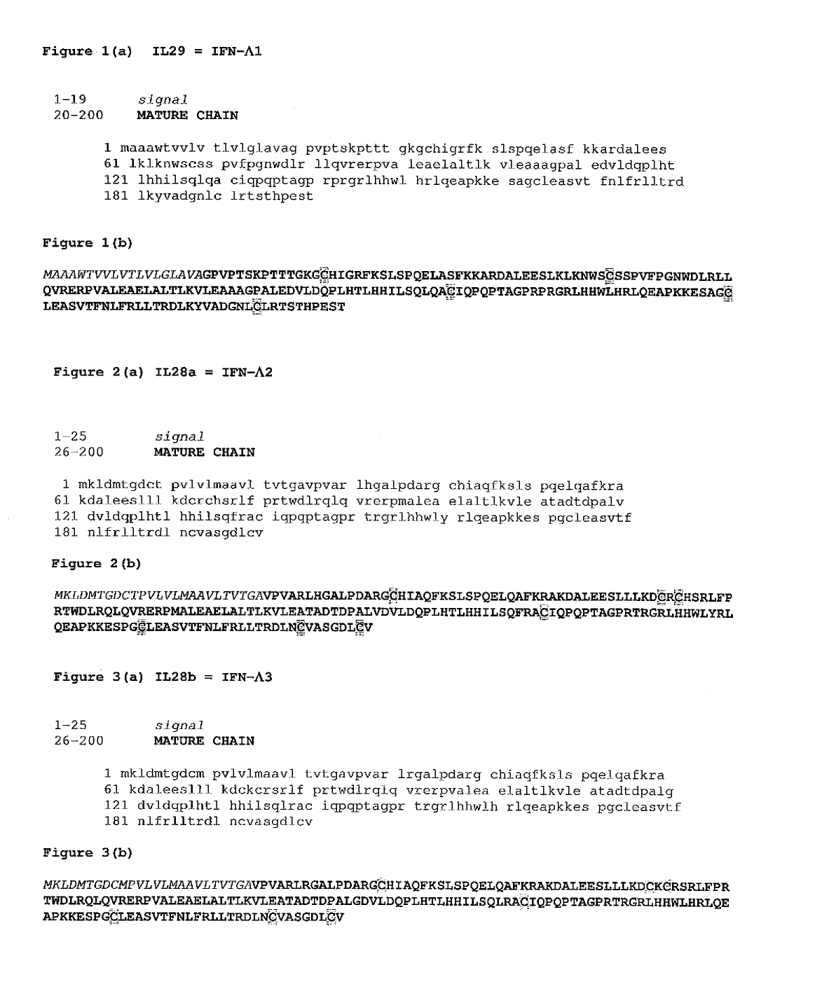

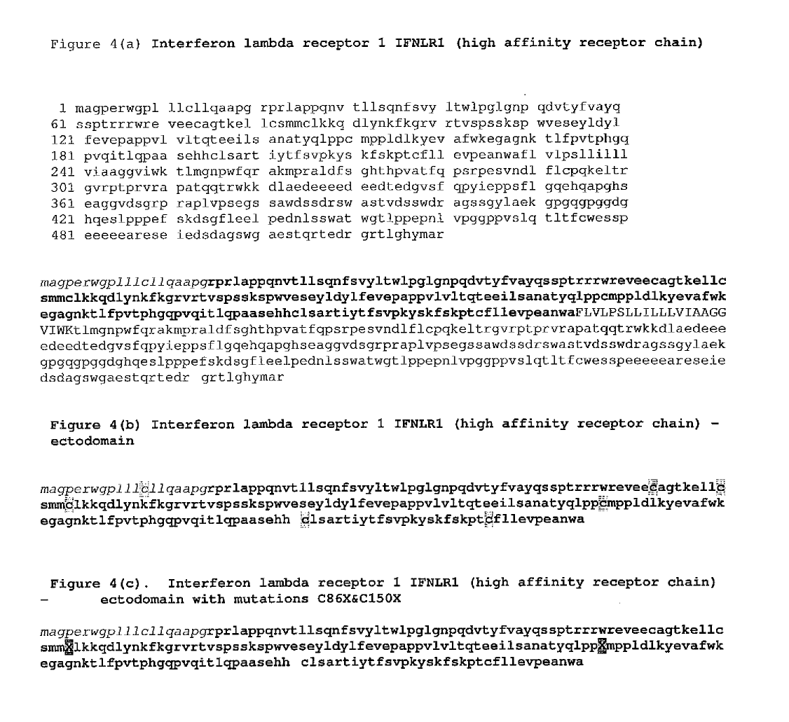

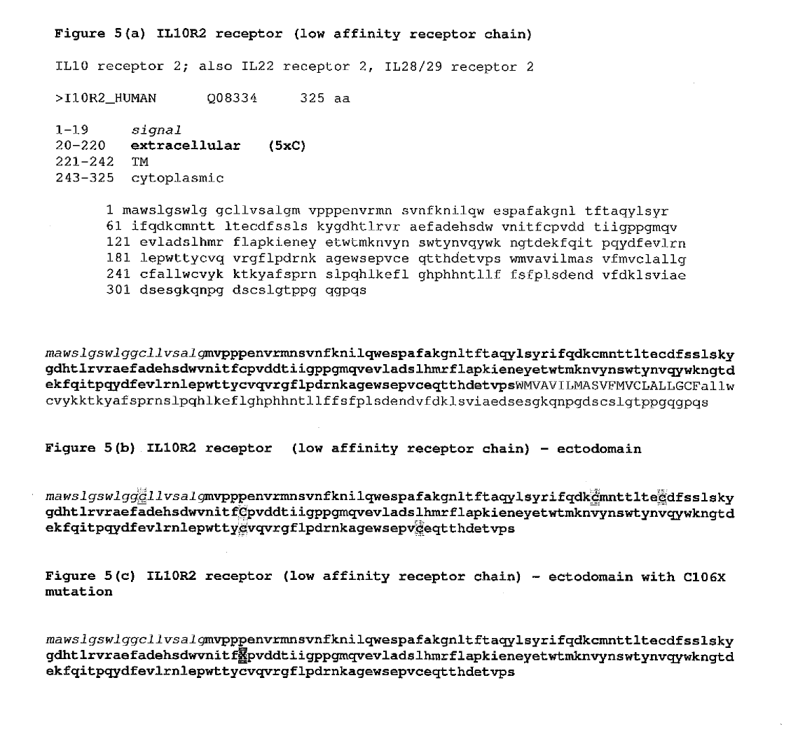

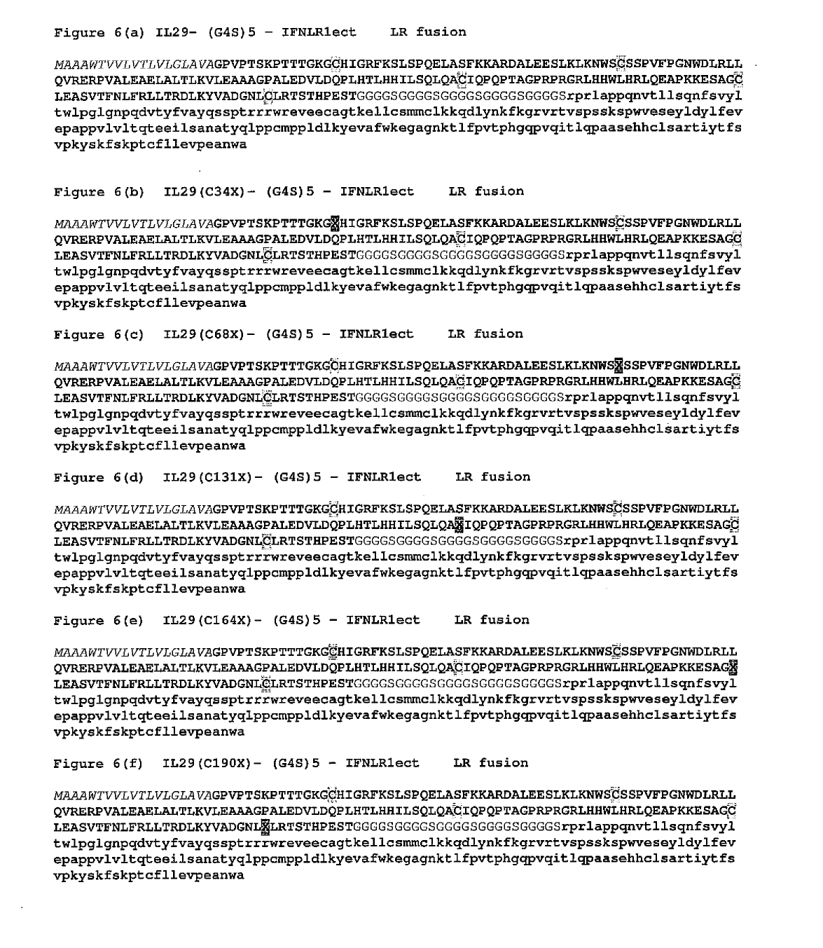

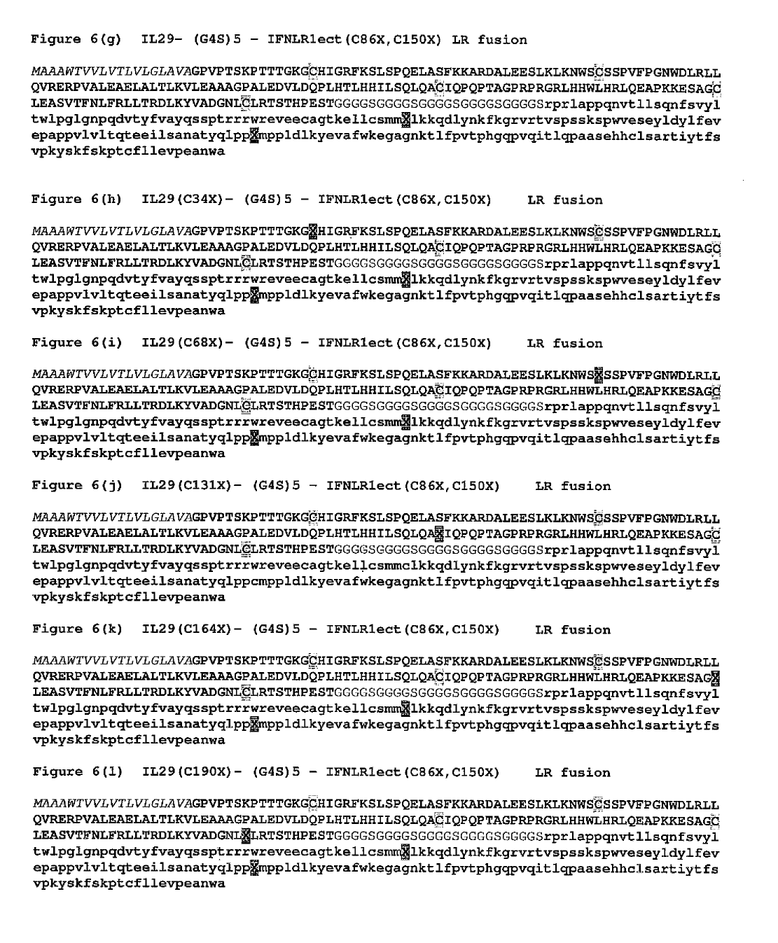

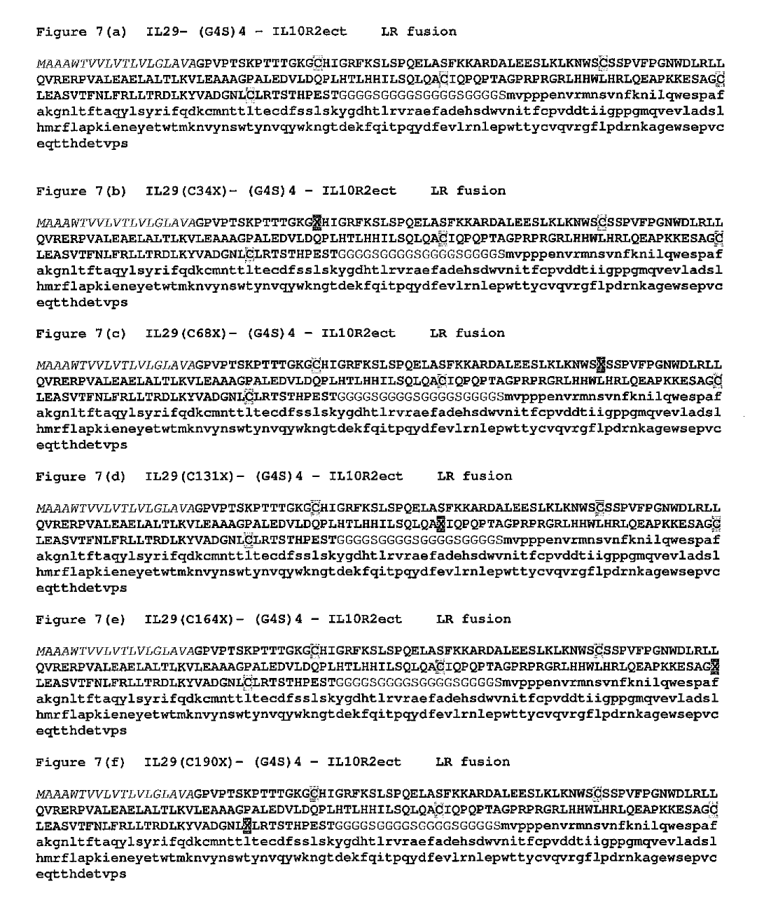

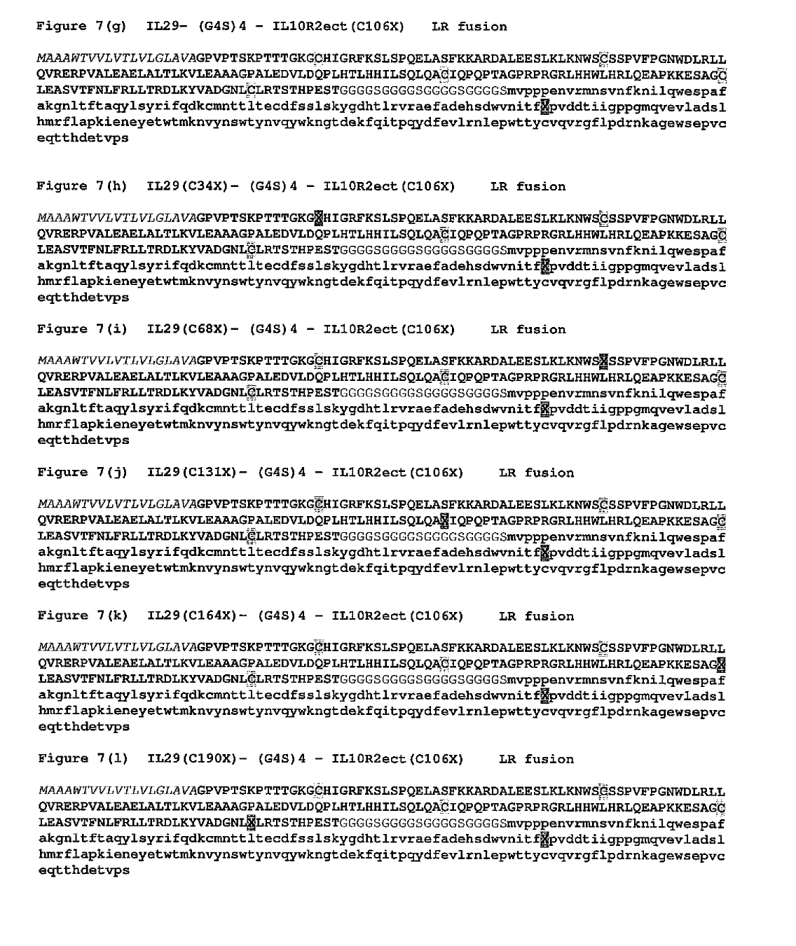

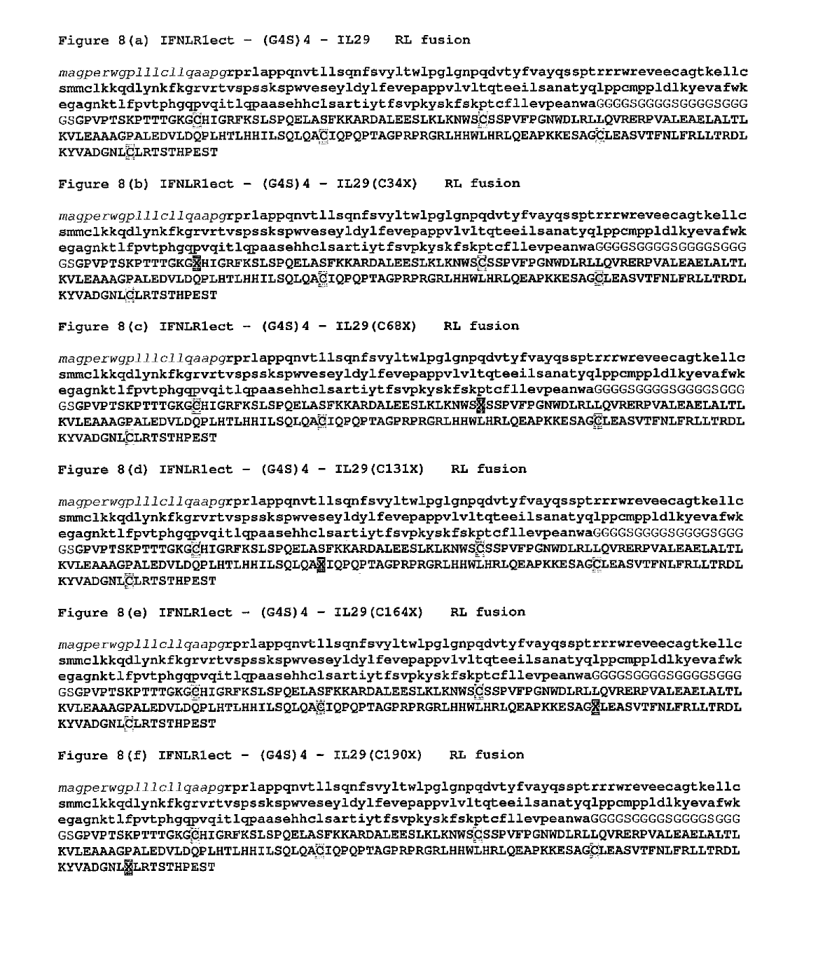

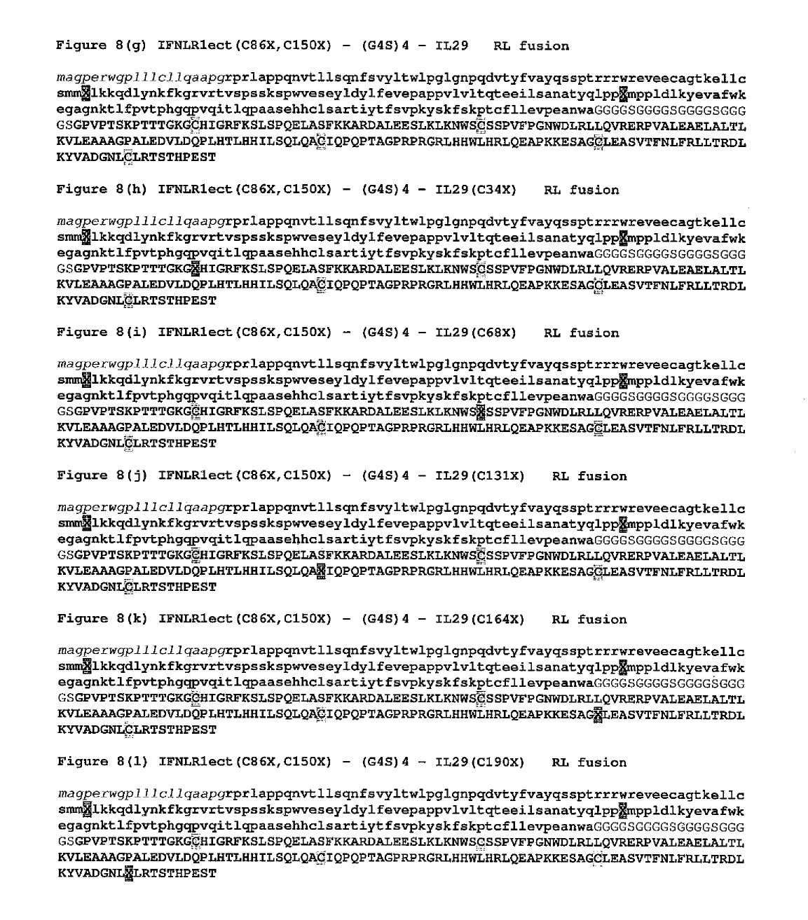

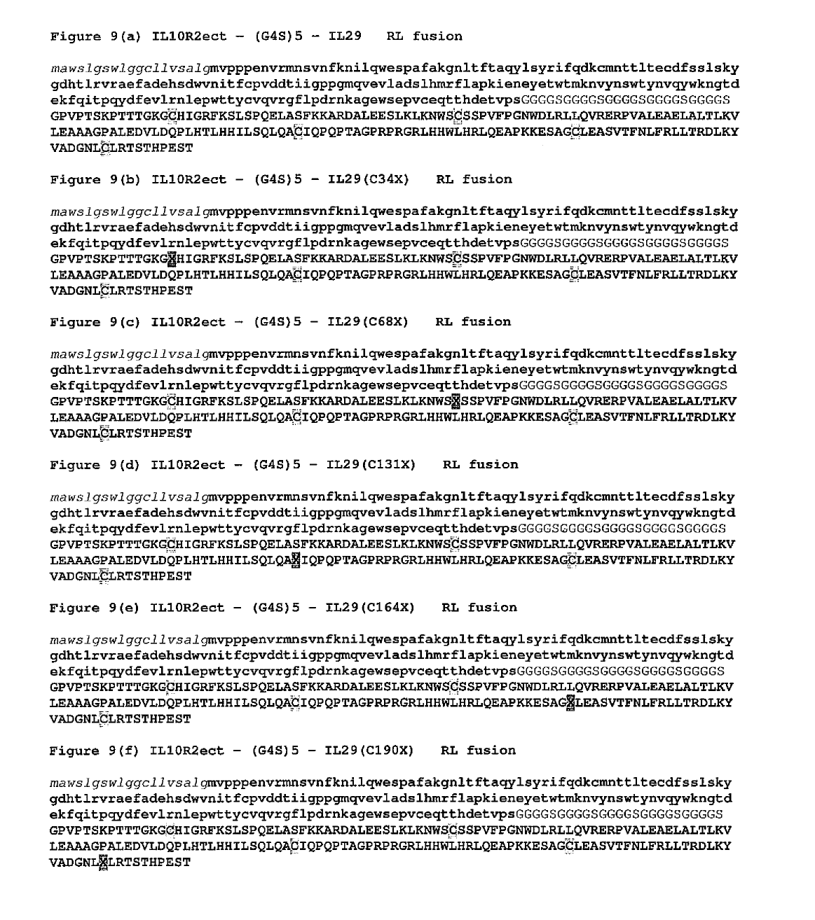

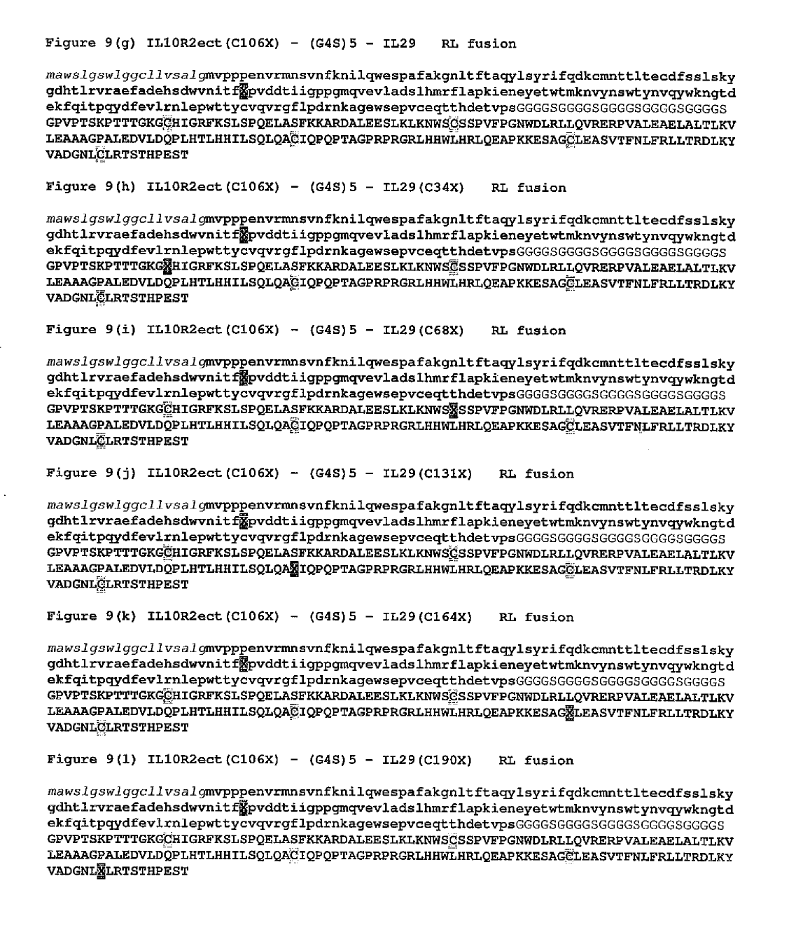

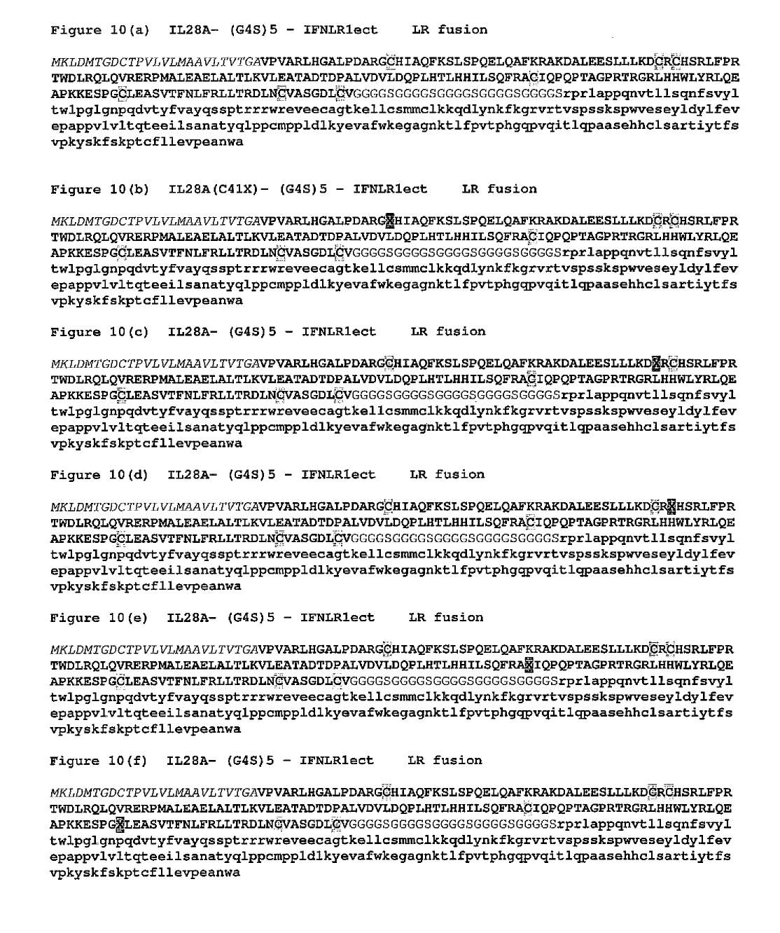

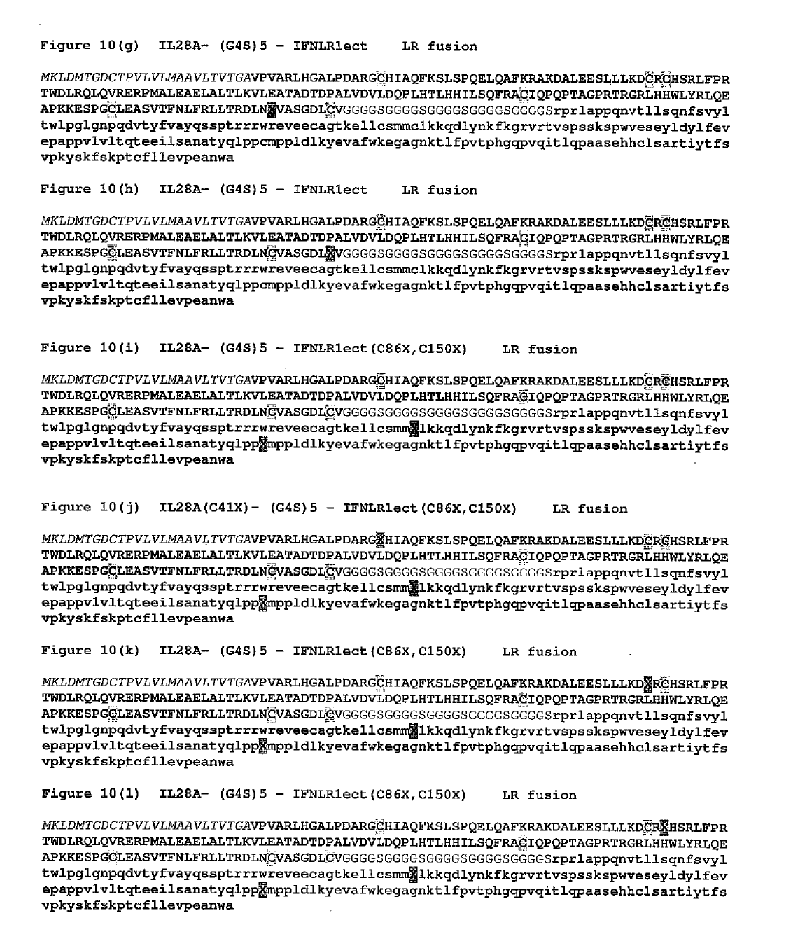

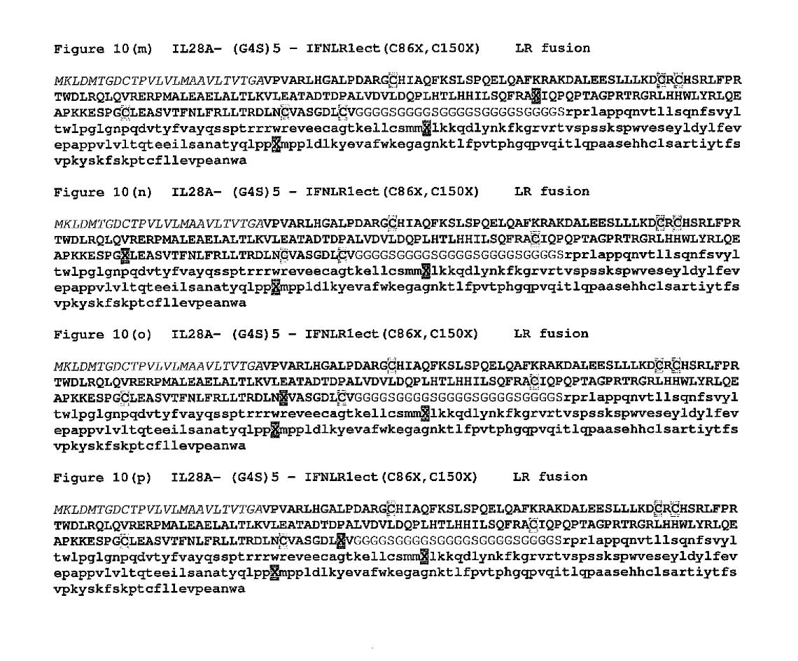

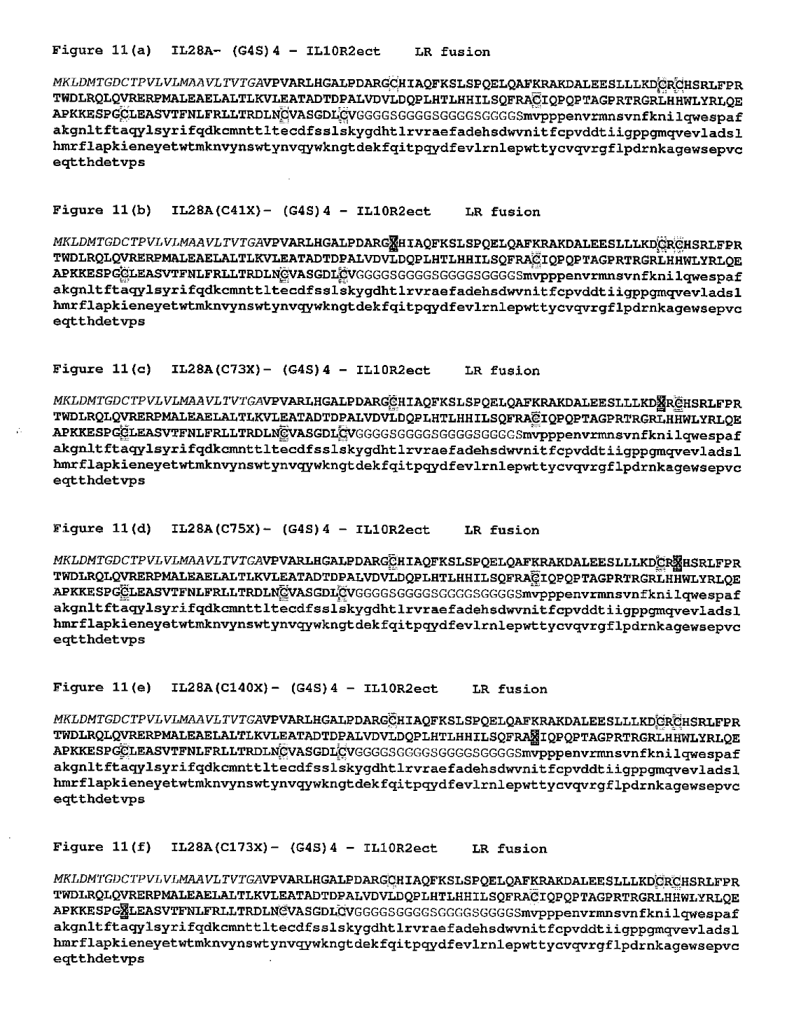

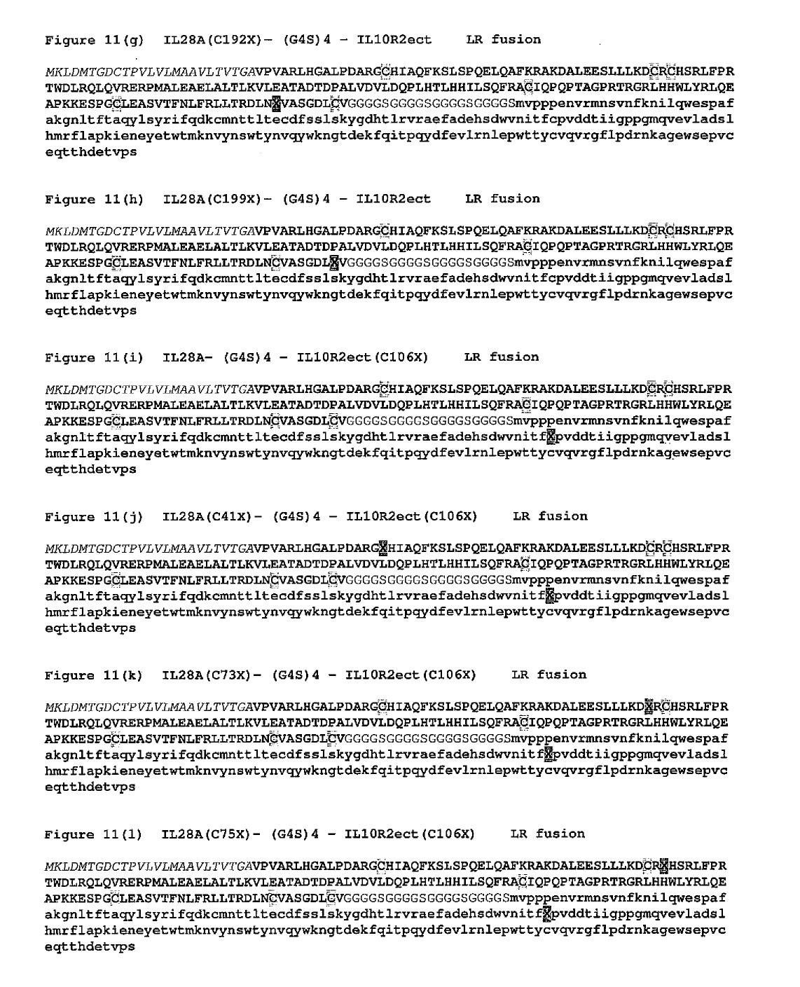

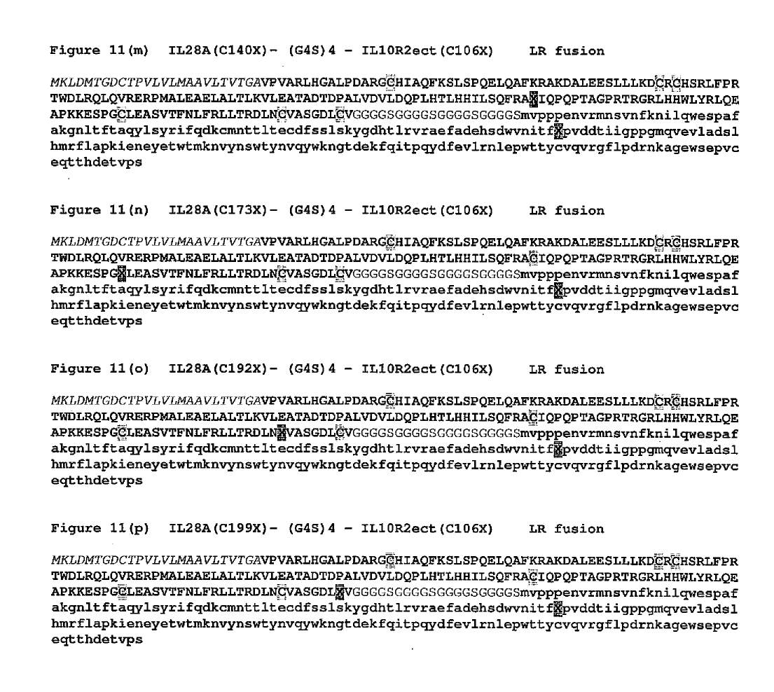

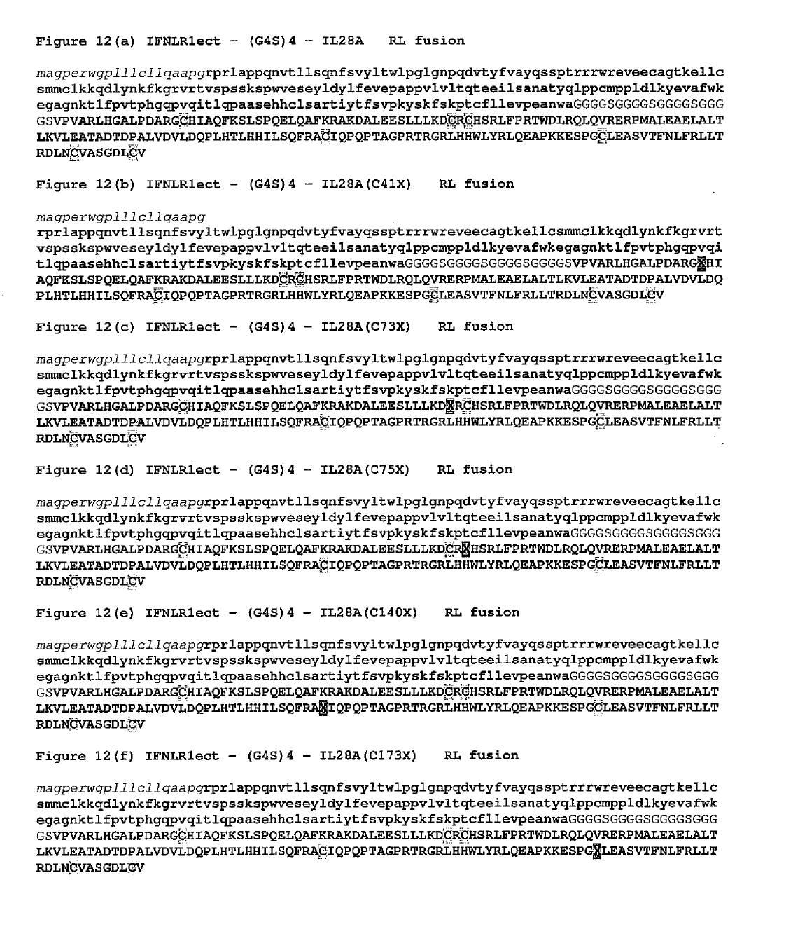

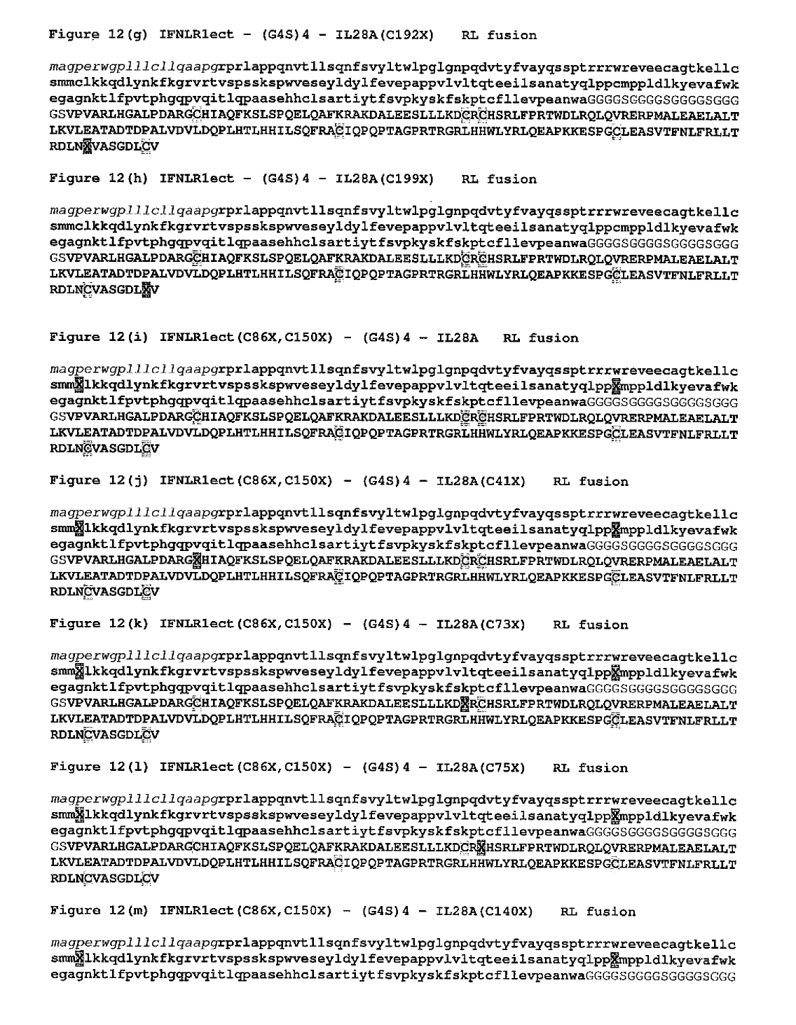

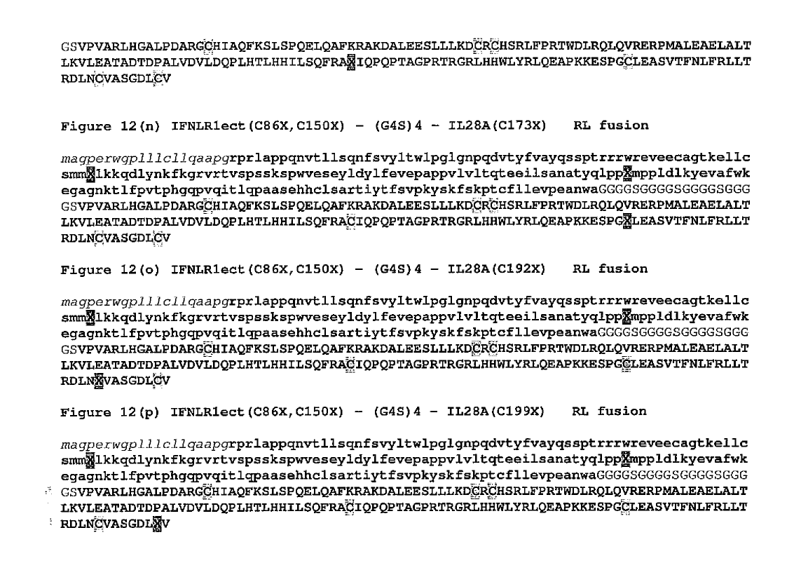

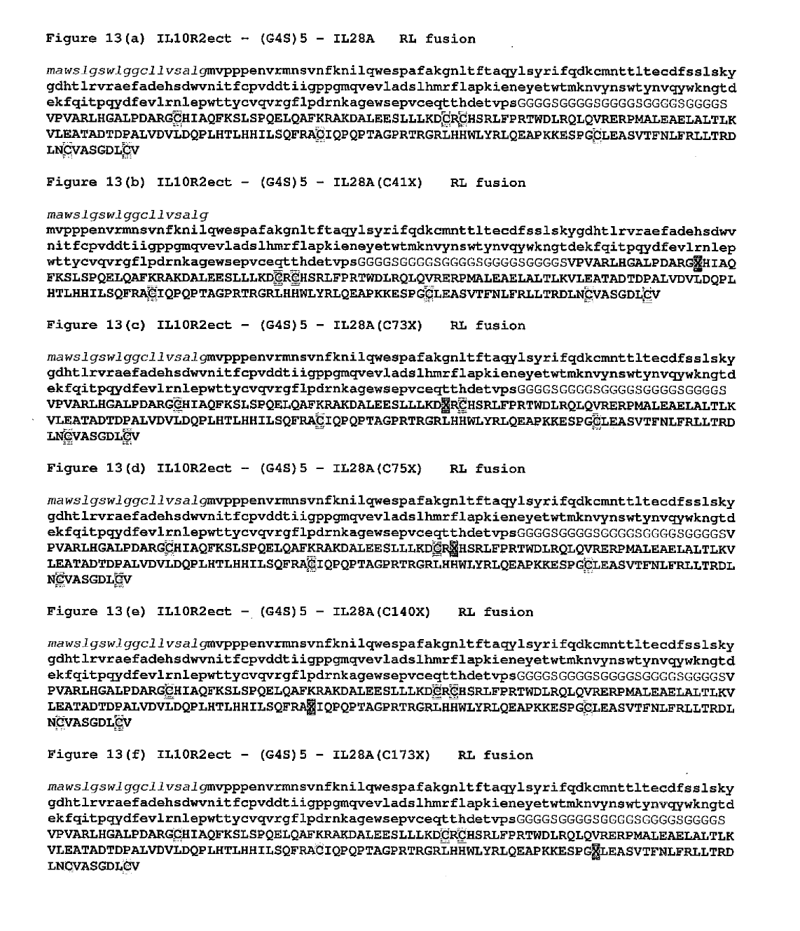

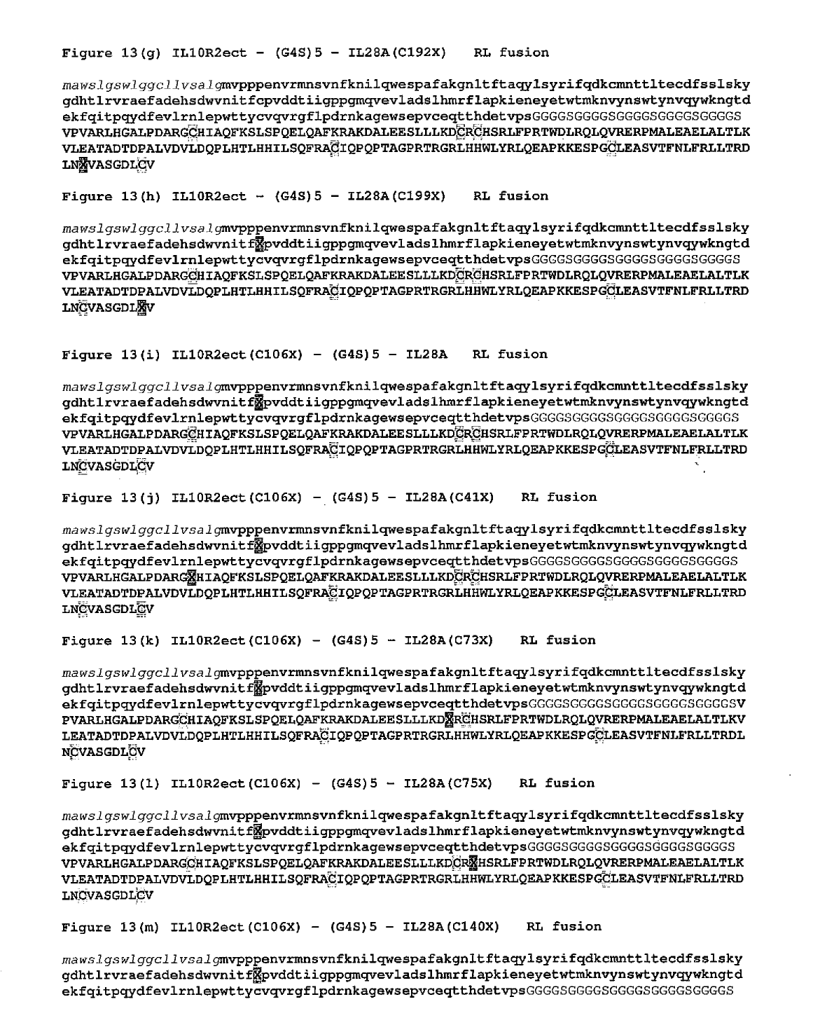

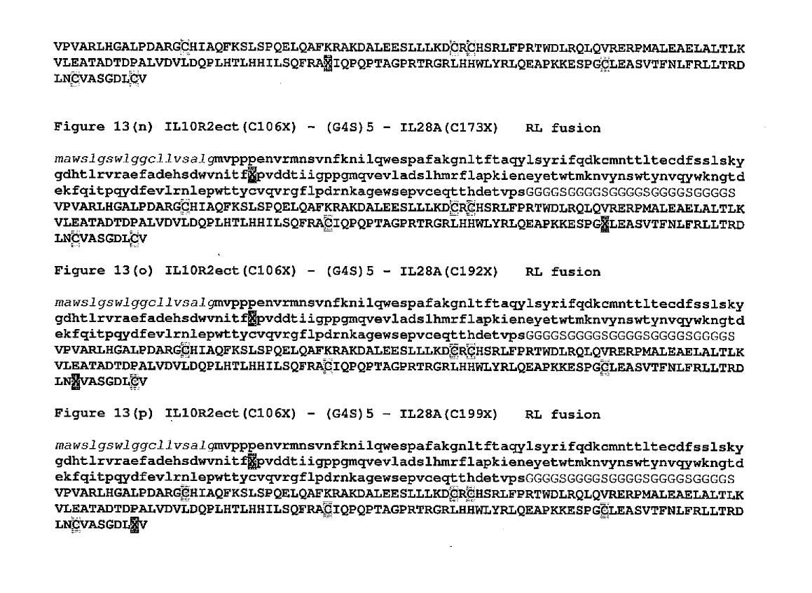

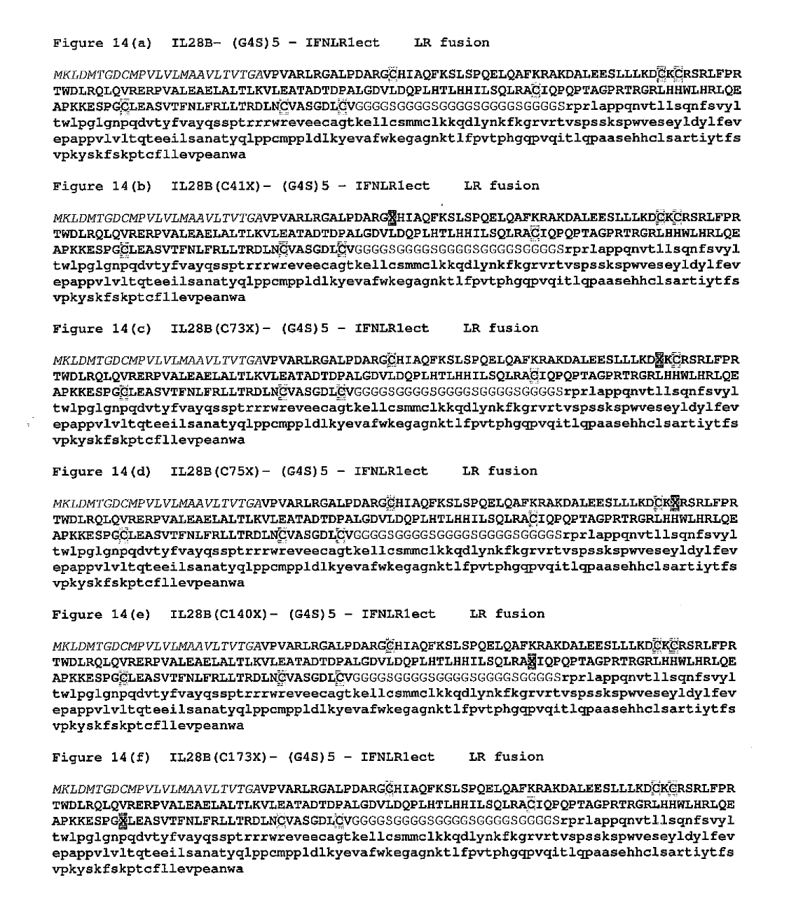

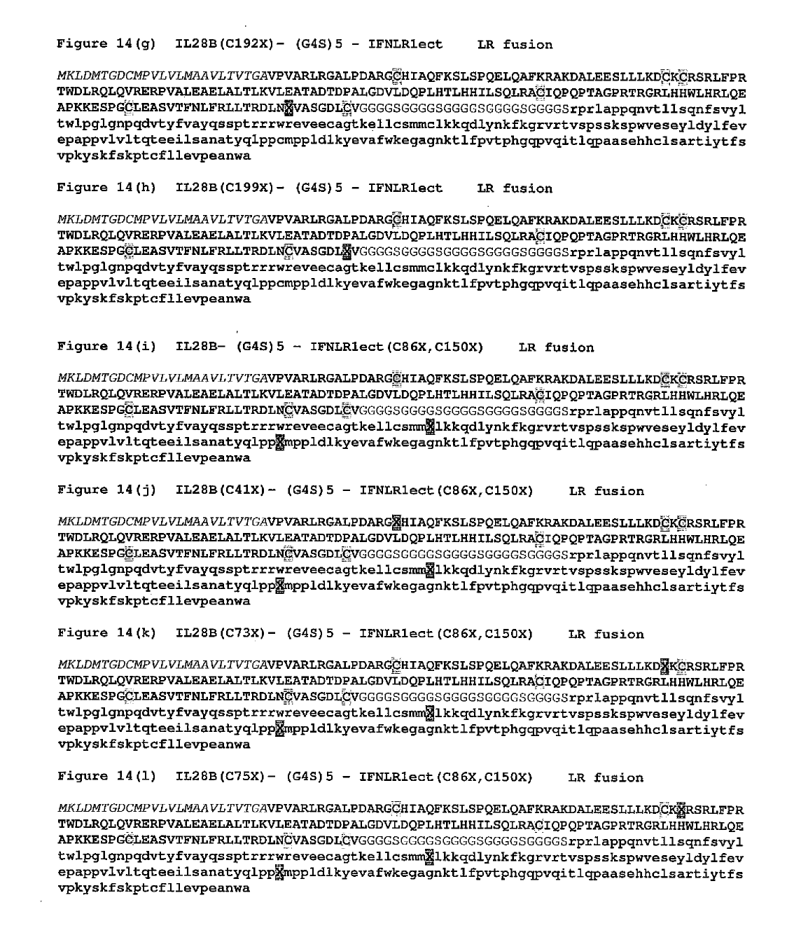

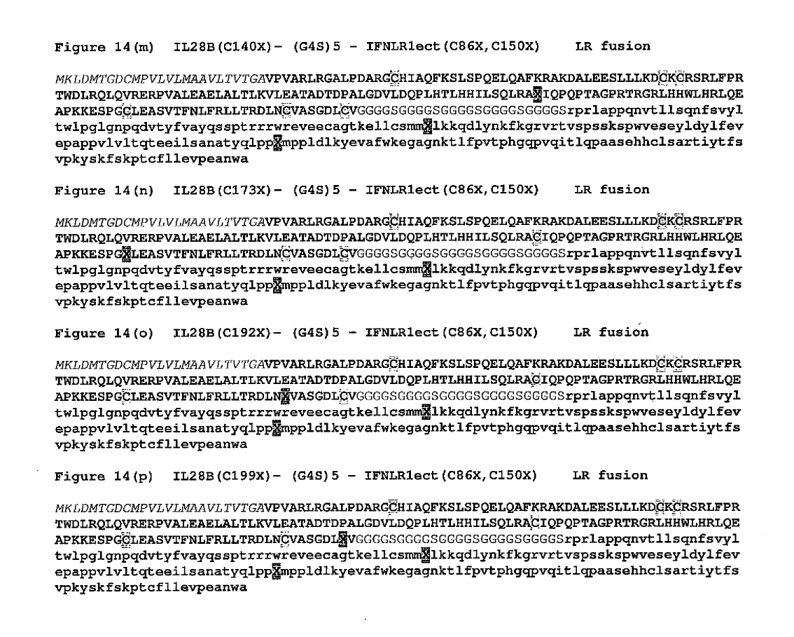

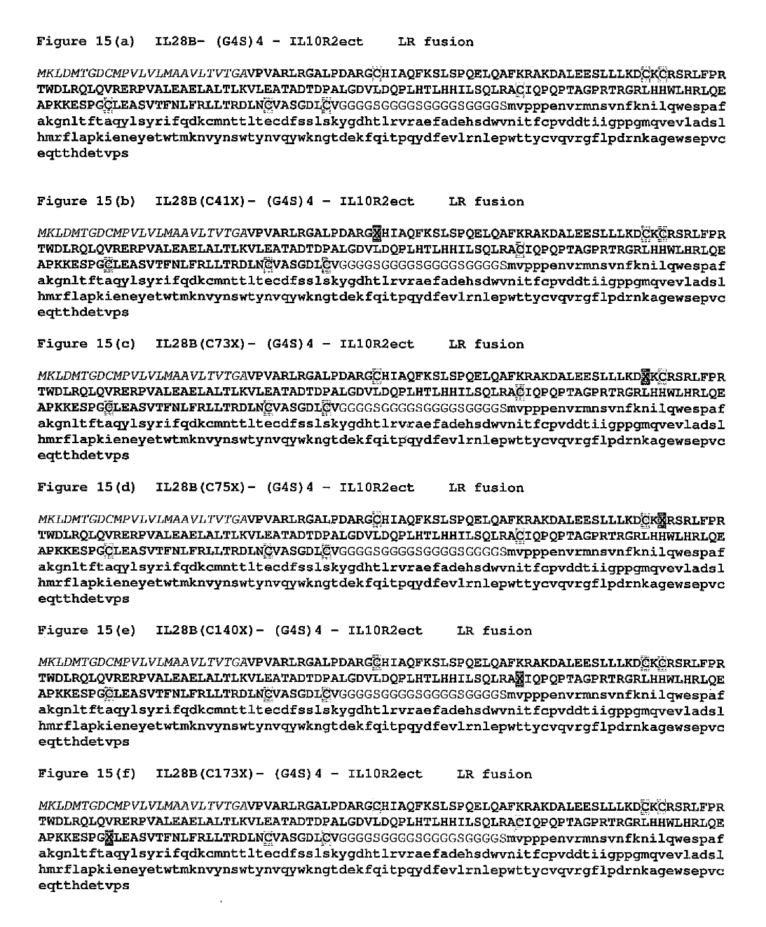

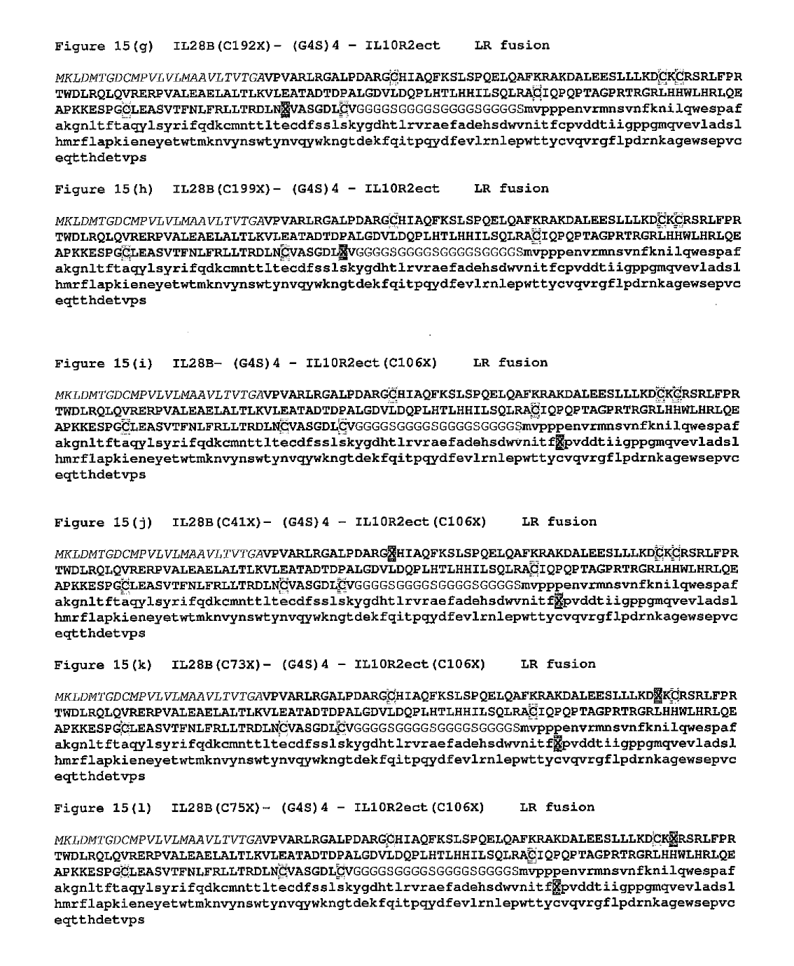

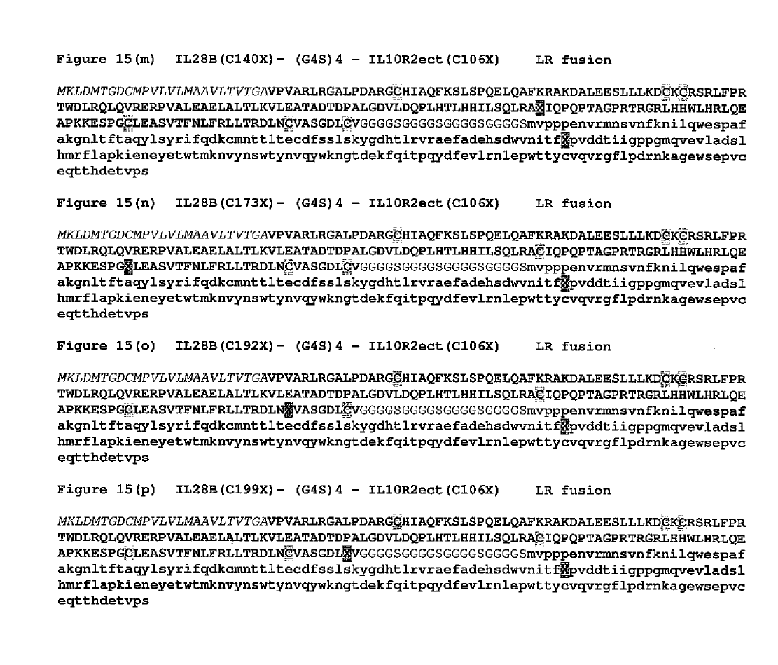

### DETAILED DESCRIPTION

### Materials and Methods

### Interferon Bioassay

Commercially available bioassays (see, SBH Sciences Inc., Nattick, Mass. and Biocpmare Inc.) can be used to test interferon. In addition, methods that assay the activity of interferon are described in Lleonart et al. (Nature Biotech (1990) 8: 1263-1267); Sedmk and Grossberg (J. Gen. Virology (1973) 21: 1-7); and Baumgarth and Kelso (J. Virology (1996) 70(7): p 4411-4418).

### Immunological Testing

Immunoassays that measure the binding of ligand or receptor to polyclonal and monoclonal antibodies are known in the art. Commercially available antibodies are available to detect the ligand or receptor in samples and also for use in competitive inhibition studies (for example, Abcam PLC., Cambridge, UK)

### Recombinant Production of Fusion Proteins

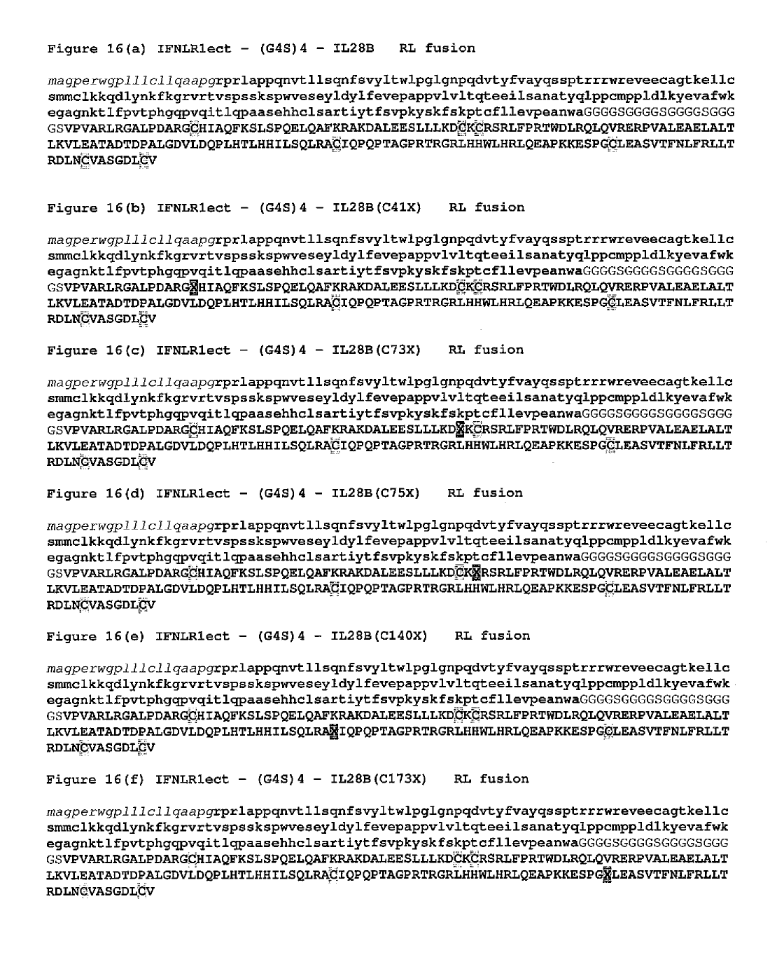

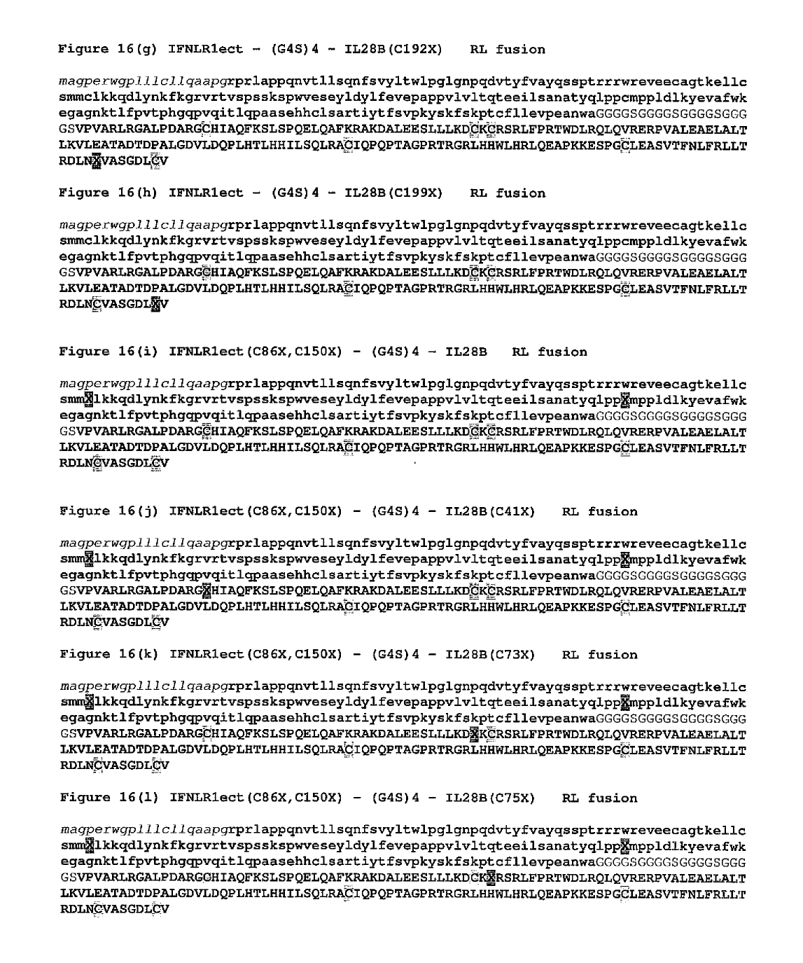

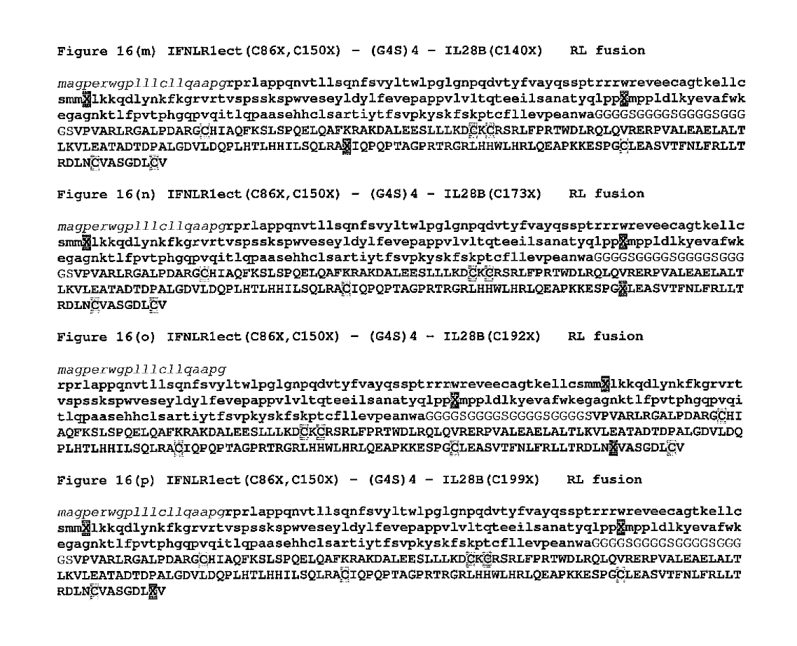

The components of the fusion proteins were generated by PCR using primers designed to anneal to the ligand or receptor and to introduce suitable restriction sites for cloning into the target vector (FIG. 16a). The template for the PCR comprised the target gene and was obtained from IMAGE clones, cDNA libraries or from custom synthesised genes. Once the ligand and receptor genes with the appropriate flanking restriction sites had been synthesised, these were then ligated either side of the linker region in the target vector (FIG. 16b). The construct was then modified to contain the correct linker without flanking restriction sites by the insertion of a custom synthesised length of DNA between two unique restriction sites either side of the linker region, by mutation of the linker region by ssDNA modification techniques, by insertion of a primer duplex/multiplex between suitable restriction sites or by PCR modification (FIG. 16c).

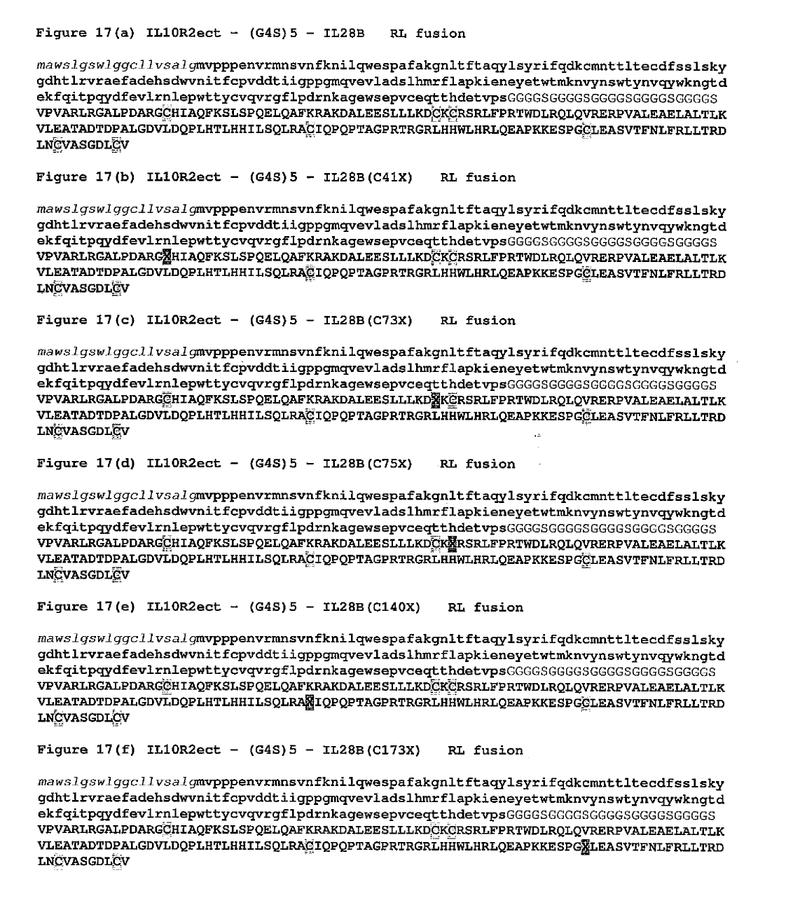

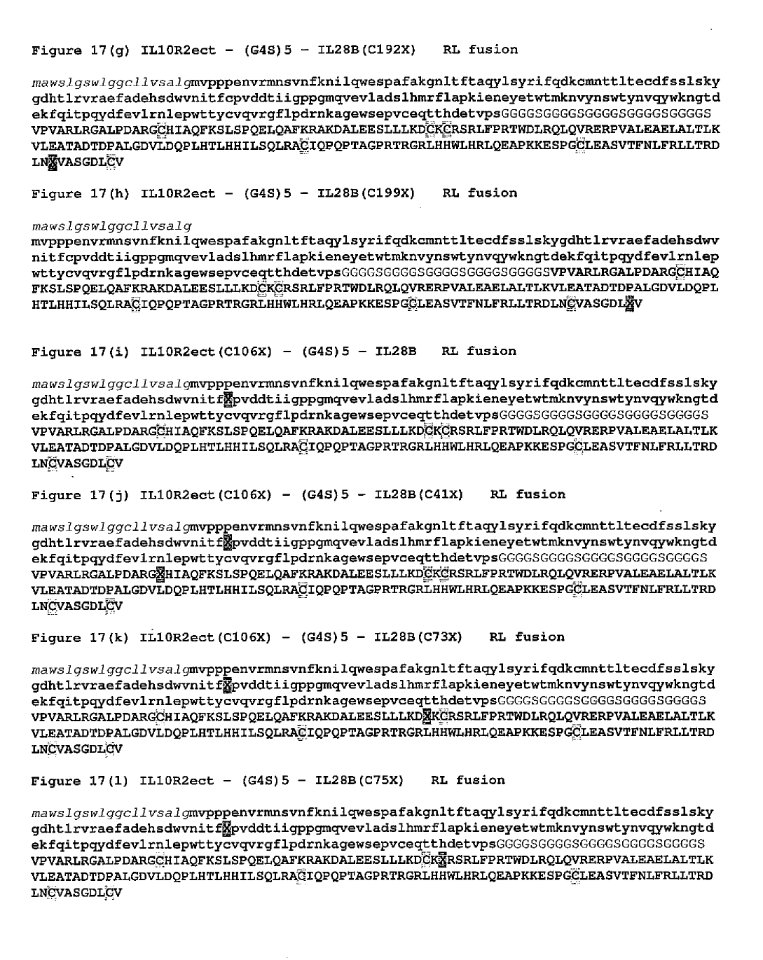

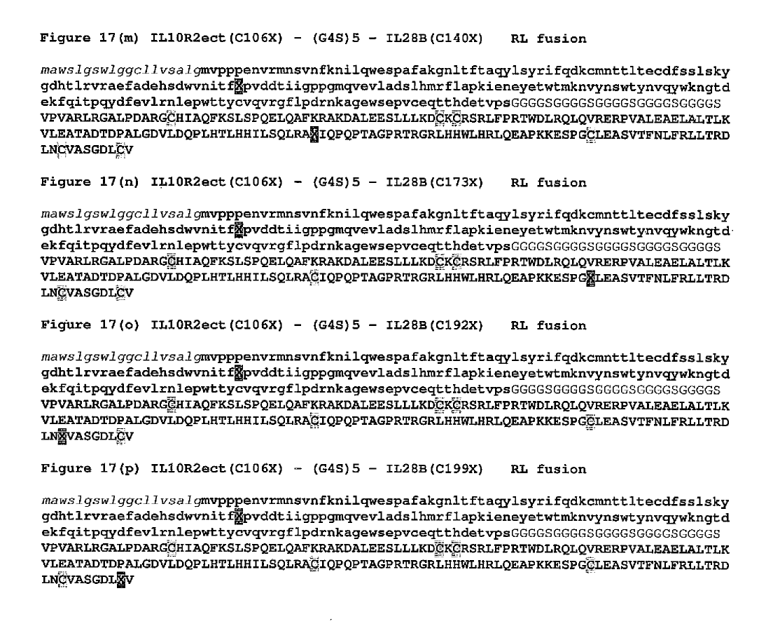

Alternatively, the linker with flanking sequence, designed to anneal to the ligand or receptor domains of choice, was initially synthesised by creating an oligonucleotide duplex and this processed to generate double-stranded DNA (FIG. 17a). PCRs were then performed using the linker sequence as a “megaprimer”, primers designed against the opposite ends of the ligand and receptor to which the “megaprimer” anneals to and with the ligand and receptor as the templates. The terminal primers were designed with suitable restriction sites for ligation into the expression vector of choice (FIG. 17b).

### Expression and Purification of Fusion Proteins

Expression was carried out in a suitable system (e.g. mammalian CHO cells, E. coli) and this was dependant on the vector into which the LR-fusion gene was generated. Expression was then analysed using a variety of methods which could include one or more of SDS-PAGE, Native PAGE, western blotting, ELISA.

Once a suitable level of expression was achieved the RL-fusions were expressed at a larger scale to produce enough protein for purification and subsequent analysis.

Purification was carried out using a suitable combination of one or more chromatographic procedures such as ion exchange chromatography, hydrophobic interaction chromatography, ammonium sulphate precipitation, gel filtration, size exclusion and/or affinity chromatography (using nickel/cobalt-resin, antibody-immobilised resin and/or ligand/receptor-immobilised resin).

Purified protein was analysed using a variety of methods which could include one or more of Bradford's assay, SDS-PAGE, Native PAGE, western blotting, ELISA.

### Characterisation of LR-Fusions

Denaturing PAGE, native PAGE gels and western blotting were used to analyse the fusion polypeptides and western blotting performed with antibodies non-conformationally sensitive to the LR-fusion. Native solution state molecular weight information can be obtained from techniques such as size exclusion chromatography using a Superose G200 analytical column and analytical ultracentrifugation.

Construction of Chimeric clones

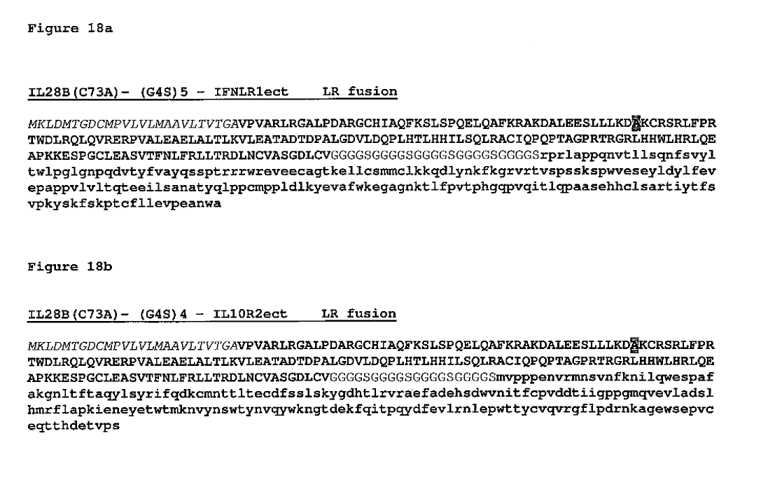

All clones were ligated using the restriction enzymes Nhe1/HindIII, into the mammalian expression plasmid pSecTag-link. Clones were attached to the secretion signal for human interferon for efficient secretion into cell media. The whole gene for a7B1 [FIG. 18a; SEQ ID NO: 186)] was cloned using gene synthesis and cloned into the mammalian expression vector pSecTag-link to form pIFNsecTag-a7B1.

Mammalian Expression of IFN Chimeric clones

A mammalian expression system has been established using a modification of the Invitrogen vector pSecTag-V5/FRT-Hist.

### Invitrogen's Flp-In System

This system allows for the rapid generation of stable clones into specific sites within the host genome for high expression. This can be used with either secreted or cytoplasmic expressed proteins. Flp-In host cell lines (flp-In CHO) have a single Flp recombinase target (FRT) site located at a transcriptionally active genomic locus

Stable cell lines are generated by co-transfection of vector (Containing FRT target site) and pOG44 (a plasmid that transiently expresses flp recombinase) into Flp-In cell line. Selection is with Hygromycin B. There is no need for clonal selection since integration of DNA is directed.

Culturing Flp-In Cell lines: followed manufactures instruction using basic cell culture techniques.

### Stable Transfection of CHO Flp-In Cells Using Fugene-6

The day before transfection, CHO Flp-In cells were seeded at 6×10E5 per 100 mm petri dish in a total volume of 10 ml of Hams F12 media containing 10% (v/v) Fetal Calf Serum, 1% Penicillin/streptomycin and 4 mM L-glutamine. The next day added 570 μl of serum free media (containing no antibiotics) to a 1.5 ml polypropylene tube. 30 μl of fugene-6 was then added and mixed by gentle rolling. A separate mix of plasmids was set up for each transfection which combined 2 μg plasmid of interest with 18 μg pOG44 (plasmid contains recombinase enzyme necessary for correct integration of plasmid into host genome). Control plate received no plasmid. This was mixed with fugene-6 by gentle rolling, left @ RmT for 15 minutes, then applied drop-wise to the surface of the each petri dish containing CHO Flp-In cells in F12 media+10% FCS. The plates were gently rolled to ensure good mixing and left for 24 hrs @ 37° C./5% CO2. The next day media was exchanged for selective media containing hygromycin B @ 600 ug/ml. Cells were routinely kept at 60% confluency or less. Cells were left to grow in the presence of 600 ug/ml hygromycin B until control plate cells (non transfected cells) had died (i.e. no hygromycin resistance).

### SDS-PAGE Analysis

Testing Expression from Stable CHO Cell Lines

Confluent CHO Flp-In cell lines expressing the protein of interest were grown in 75 cm2 flasks for approximately 3-4 days in serum free media, at which point samples were taken and concentrated using acetone precipitation. Samples were mixed with an equal volume of laemmli loading buffer in the presence or absence of 25 mM DTT and boiled for 5 minutes. Samples were analysed by SDS-PAGE and transferred to a PVDF membrane. After blocking in 5% (w/v) Milk protein in PBS-0.05% (v/v) Tween 20, sample detection was carried out using a specific anti-IGF-1 antibody together with a Horse Radish Peroxidase (HRP) conjugated secondary antibody. Visualisation was by chemiluminesence on photographic film using an HRP detection kit.

Testing Expression from Transient Tansfections of CHO FlpIn

CHO Flp-In cells were seeded at 0.25×10E6 cells per well of a 6 well plate in a total volume of 2 ml media (DMEM, F12, 10% FCS+P/S+L-glutamine+Zeocin). Cells were left to grow o/n. Cells were then transfected using either TranslT-CHO Reagent (Mirus) or fugene-6 at the specified reagent ratios stated in table 1. Control transfections were set up using 1B7stop (GH containing chimeric molecule). Briefly, if using TransIT reagent, 200 ul of Serum free media (OPTI MEM) was added to a 1.5 ml eppendorff per transfection followed by 2 ug DNA. The tubes were left for 15 minutes at RmT. 1 ul of CHO Mojo Reagent was then added, mixed and left for a further 15 minutes. Media was changed to serum free and the transfection mix pippetted dropwise onto the surface of the appropriate well. Briefly, if using Fugene-6 reagent, 94 ul of Serum free media (OPTI MEM) was added to a 1.5 ml seppendorff per transfection followed by 2 ug DNA. The tubes were left for 15 minutes at RmT. Transfection mix was then pippetted drop wise onto the surface of the appropriate well containing serum free media. All plate were left @ 37° C./5% CO₂ for 2-3 days

### Statistics

Two groups were compared with a Student's test if their variance was normally distributed or by a Student-Satterthwaite's test if not normally distributed. Distribution was tested with an F test. One-way ANOVA was used to compare the means of 3 or more groups and if the level of significance was p<0.05 individual comparisons were performed with Dunnett's tests. All statistical tests were two-sided at the 5% level of significance and no imputation was made for missing values.

### EXAMPLE 1

All clones were ligated using the restriction enzymes NheI/HindIII, into the mammalian expression plasmid pSecTag-link. Clones were attached to the secretion signal for human IFNI3 for efficient secretion into cell media. The whole genes for 13E1 and 13F1 were cloned using gene synthesis and cloned into the mammalian expression vector pSecTag-link to form pIFNI3SecTag 13E1 and pIFNI3SecTag13F1.

### EXAMPLE 2

A mammalian expression system has been established using a modified Invitrogen vector pSecTag-V5/FRT-Hist. This vector is used in Invitrogen's FIp-In system to direct integration of the target gene into the host cell line, allowing rapid generation of stable clones into specific sites within the host genome for high expression. Culturing FIp-In Cell lines: followed manufacturer's instruction using basic cell culture techniques.

Stable cell lines were generated in 6-well plates using Fugene-6 as the transfection reagent. The CHO FIp-In cells were co-transfected with the expression vector and pOG44, a plasmid that expresses flp recombinase, an enzyme which causes the recombination of the LR-fusion gene into a “hot-spot” of the cell chromosome. Hygromycin B was used to select for cells with positive recombinants.

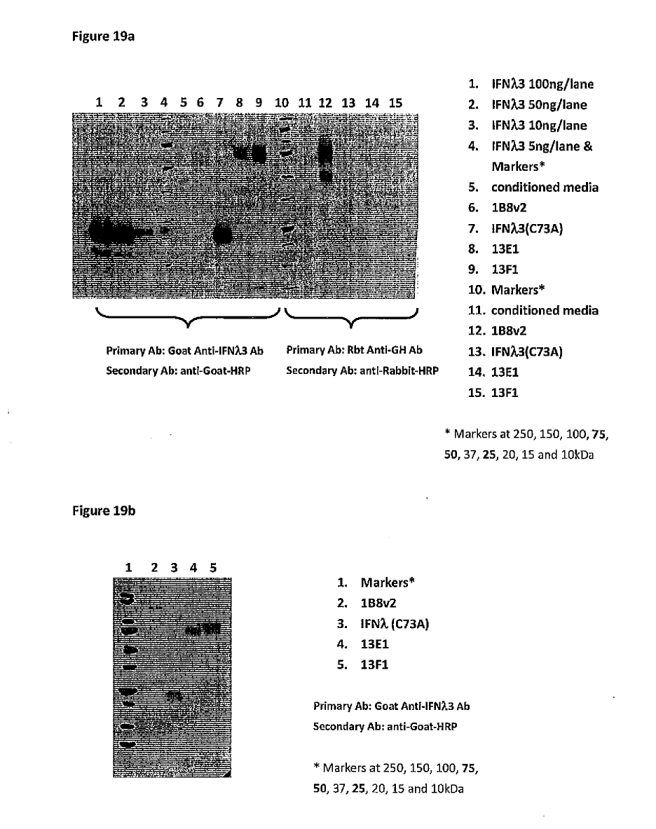

Once the stable cell lines had been established they were grown on 75 cm² culture plates, at a confluency of 50-70% the media was changed to serum free media. The cultures were incubated for a further 2-4 days after which media samples were taken. These were run on 13% SDS-PAGE gels and transferred to PVDF membrane for immuno-blotting. After blocking in 5% (w/v) milk protein in PBS+0.05% (v/v) Tween 20, sample detection was carried out using a specific anti-IL28B antibody together with a Horse Radish Peroxidase (HRP) conjugated secondary antibody. Visualisation was by chemiluminesence on photographic film using an HRP detection kit. The transient and stable expression of fusion protein 13E1 and 13F1 as represented by the amino acid sequence shown in FIGS. 18a and 18b (SEQ ID NO: 186 and 187) are illustrated in FIG. 19a and FIG. 19b respectively.

1. A fusion polypeptide comprising: the amino acid sequence of an interferon λ, or an amino acid sequence variant thereof, linked, directly or indirectly, to the binding domain of an interferon λ receptor, or an amino acid sequence variant thereof.

2. A fusion polypeptide according to claim 1 wherein the variant polypeptide varies by substitution of at least one cysteine amino acid residue.

3. A fusion polypeptide according to claim 2 wherein said cysteine amino acid residue is substituted for an amino acid selected from the group consisting of: aspartic acid, glutamic acid, phenylalanine, glycine, histidine, isoleucine, lysine, leucine, methionine, proline, glutamine, arginine, threonine, valine, tryptophan and tyrosine.

4. A fusion polypeptide according to claim 3 wherein said cysteine substitution is for serine, alanine or asparagine.

5. A fusion polypeptide according to claim 1 wherein said interferon λ is linked to the binding domain of the interferon λ receptor by a peptide linker molecule.

6. A fusion polypeptide according to claim 5 wherein said peptide linker molecule comprises at least 1, 2, 3, 4, 5 or 6 copies of the peptide Gly Gly Gly Gly Ser (SEQ ID NO: 188).

7. A fusion polypeptide according to claim 6 wherein said peptide linker molecule consists of 4 or 5 copies of the peptide Gly Gly Gly Gly Ser (SEQ ID NO: 188).

8. A fusion polypeptide according to claim 5 wherein said peptide linker molecule comprises or consists of one copy of the glycosylation motif Asn-Xaa-Ser or Asn-Xaa-Thr where X is any amino acid except proline.

9. A fusion polypeptide according to claim 8 wherein said peptide linker molecule comprises at least 5 amino acid residues.

10. A fusion polypeptide according to claim 9 wherein said peptide linker molecule comprises 5-50 amino acid residues.

11. A fusion polypeptide according to claim 10 wherein said peptide linker molecule comprises at least one copy of the motif (Xaa₁ Xaa₂ Xaa₃ Xaa₄ Xaa₅; SEQ ID NO: 189) wherein said motif comprises the glycosylation motif Asn-Xaa-Ser or Asn-Xaa-Thr.

12. A fusion polypeptide according to claim 1 wherein said polypeptide does not comprise a peptide linker molecule and is a direct fusion of interferon λ and the interferon λ binding domain of the interferon λ receptor.

13. A fusion polypeptide according to claim 1 wherein said fusion polypeptide comprises an amino acid sequence as represented in SEQ ID NO: 1-3, wherein said polypeptide optionally includes a signal sequence.

14. A fusion polypeptide according to claim 13 wherein interferon λ is represented by SEQ ID NO: 1.

15. A fusion polypeptide according to claim 14 wherein said interferon λ amino acid sequence variant is a modification of amino acid residue cysteine 190 in SEQ ID NO: 1.

16. A fusion polypeptide according to claim 15 wherein cysteine 190 is modified by an amino acid substitution.

17. A fusion polypeptide according to claim 16 wherein said substitution is cysteine 190 for a serine amino acid.

18. A fusion polypeptide according to claim 14 wherein said amino acid sequence variant is a modification of amino acid residue cysteine 34 in SEQ ID NO: 1.

19. A fusion polypeptide according to claim 14 wherein cysteine 34 is modified by an amino acid substitution.

20. A fusion polypeptide according to claim 19 wherein said substitution is cysteine 34 for a serine amino acid.

21. A fusion polypeptide according to claim 14 wherein said interferon λ amino acid sequence variant is a modification of amino acid residue 190 and amino acid residue 34.

22. A fusion polypeptide according to claim 1 wherein interferon λ is represented by SEQ ID NO: 2 or 3.

23. A fusion polypeptide according to claim 22 wherein interferon λ amino acid sequence variant is a modification of amino acid residue cysteine 73 in SEQ ID NO: 2 or 3.

24. A fusion polypeptide according to claim 23 wherein cysteine 73 is modified by an amino acid substitution.

25. A fusion polypeptide according to claim 24 wherein said substitution is cysteine 73 for a serine amino acid.

26. A fusion polypeptide according to claim 24 wherein said substitution is cysteine 73 for an alanine amino acid.

27. A fusion polypeptide according to claim 22 wherein interferon λ amino acid sequence variant is a modification of amino acid residue cysteine 75 in SEQ ID NO: 2 or 3.

28. A fusion polypeptide according to claim 27 wherein said modification is a substitution of cysteine 75 for a serine amino acid.

29. A fusion polypeptide according to claim 1 wherein the interferon binding domain of an interferon receptor is an interferon λ receptor binding domain comprising SEQ ID NO: 4, 5 or 6, wherein said polypeptide optionally includes a signal sequence.

30. A fusion polypeptide according to claim 29 wherein interferon λ is linked to an interferon λ binding domain of an interferon λ receptor and wherein said interferon λ is positioned amino terminal to said binding domain in said fusion polypeptide.

31. A fusion polypeptide according to claim 30 wherein interferon λ is linked to an interferon λ binding domain of an interferon λ receptor and wherein said interferon is positioned carboxyl-terminal to said binding domain in said fusion polypeptide.

32. A fusion polypeptide according to claim 1 wherein said fusion polypeptide comprises an amino acid sequence as represented in SEQ ID NO: 10-187, wherein said polypeptide optionally includes a signal sequence.

33. A homodimer consisting of two polypeptides wherein each of said polypeptides comprises:
i) a first part comprising interferon λ, or a receptor binding domain thereof; and
ii) a second part comprising at least one interferon λ binding domain or part thereof, of an interferon λ receptor.

34. A nucleic acid molecule that encodes a polypeptide according to claim 1.

35. A vector comprising a nucleic acid molecule according to claim 34.

36. A cell transfected or transformed with a vector according to claim 35.

37. A pharmaceutical composition comprising a polypeptide according to claim 1 and an excipient or carrier.

38. A composition according to claim 37 wherein said pharmaceutical composition is combined with a further therapeutic agent.

39. A method to treat a human subject suffering from a viral infection comprising administering an effective amount of a polypeptide according to claim 1.

40. A method according to claim 39 wherein said viral infection is caused by a hepatitis virus.

41. A method according to claim 40 wherein said hepatitis virus is selected from the group consisting of: hepatitis A, B, C, D or E.

42. A method to treat a human subject suffering from cancer comprising administering an effective amount of a polypeptide according to claim 1.
